(19) United States
(12) Patent Application Publication
(10) Pub. No.: US 2014/0141997 A1
(43) Pub. Date: May 22, 2014

(54) FOETAL NUCLEATED RED BLOOD CELL DETECTION

(71) Applicant: NATIONAL UNIVERSITY OF SINGAPORE, Singapore (SG)

(72) Inventors: Aniza Puteri Mahyuddin, Singapore (SG); Sukumar Ponnusamy, Singapore (SG); Priya Kadam, Singapore (SG); Mahesh Choolani, Singapore (SG)

(73) Assignee: NATIONAL UNIVERSITY OF SINGAPORE, Singapore (SG)

(21) Appl. No.: 14/129,889

(22) PCT Filed: Jun. 28, 2012
(86) PCT No.: PCT/SG2012/000229

§ 371 Date: Dec. 27, 2013

Related U.S. Application Data
(60) Provisional application No. 61/503,236, filed on Jun. 30, 2011.

(51) Int. Cl. G01N 33/569

(52) U.S. Cl. CPC G01N 33/56966
USPC 506/9; 435/7.25; 435/6.12; 435/6.11

(57) ABSTRACT

In one aspect, the invention is directed to methods of separating foetal nucleated red blood cells (FNRBCs) from a sample comprising contacting the sample with an agent that specifically recognizes CD147, thereby producing a mixture; maintaining the mixture under conditions in which a complex forms between the agent and the CD147 in the sample; and separating the complex from the mixture; thereby separating the FNRBCs from the sample. In other embodiments, the invention is directed to methods of detecting FNRBCs in a sample comprising contacting the sample with an antibody or antigen binding fragment thereof that specifically binds CD147, thereby producing a combination; maintaining the combination under conditions in which an immune complex forms between the antibody and FNRBCs present in the sample; and detecting whether the immune complex forms in the combination; wherein if the immune complex is detected then FNRBCs are present in the sample. In still other aspects, the invention further comprises methods of separating FNRBCs from anucleated RBCs, detecting prenatal disorders and/or the gender of a foetus.

### FIELD OF THE INVENTION

The present invention relates to the use of a cellular marker for detecting, isolating and/or separating at least one foetal nucleated red blood cell from a sample.

### BACKGROUND OF THE INVENTION

Prenatal diagnosis provides valuable information on the health of the unborn child and can include invasive and non-invasive methods. The first reliable genetic diagnosis using amniocentesis was performed by Steele and Bregg in 1966. Later, first trimester chorionic villus sampling (CVS) was shown to be a safe and reliable approach for earlier prenatal diagnosis. Although both are invasive techniques and harbour potential risks of foetal miscarriage they are still considered to be the gold standards of prenatal diagnosis. Hence, these are slowly being replaced by the non-invasive prenatal diagnosis (NIPD) methods wherein foetal cells/genetic material obtained from maternal circulation are being utilized for prenatal diagnosis. In particular, identification of cell-free DNA, mRNA and foetal cells in the maternal circulation made the possibility of NIPD for diagnosis of chromosomal anomalies and single gene defects of the foetus. However, the foetal genetic materials obtained from the maternal circulation are rather insufficient to provide reliable information on chromosomal abnormalities. In particular, the cell-free DNA in maternal circulation is rather insufficient to provide complete chromosomal information such as aneuploidies for diagnosis and is also expensive. On the other hand, the foetal cells are promising candidates for detecting chromosomal abnormalities but their cell numbers are very few. In particular, the utilization of foetal cells circulating in the maternal blood is both promising for detection of aneuploidies as well as in providing complete genetic information of the foetus. Here again the major limitations are their scarcity in maternal circulation and lack of efficient separation techniques. Moreover, some of these cells might persist from previous pregnancy and may not be indicative of the current foetal status.

It is well known that prenatal diagnosis enables early identification of congenital birth defects and other risk factors that impair foetal survival, which in turn helps early intervention thereby avoiding complications and relieving parent anxiety. Of the various methods that are currently available, a diagnosis on isolated human foetal erythroblasts (hFEs) and/or Foetal Nucleated Red Blood Cells (FNRBCs) in the maternal circulation would be the most reliable and non-invasive strategy. This is because FNRBCs have unique identification markers and their presence is definitely indicative of the current pregnancy and hence considered a potential candidate for early first trimester NIPD.

In particular, current methods routinely followed to obtain foetal cells for prenatal diagnosis of chromosomal and monogenic disorders (i.e. amniocentesis, chorionic villus sampling and cordocentesis) carry a small but inherent risk of miscarriage (0.5-4%). Foetal DNA from the first trimester maternal blood offers a promise of a non-invasive alternative to current prenatal diagnosis methods.

In view of the scarcity of foetal erythroblasts, to date no method has successfully identified and/or isolated foetal erythroblasts. Studies on foetal erythroblasts have relied only on heterogenous culture of cells, which may not provide accurate information in view of maternal cells or other impurities. Poor in vitro viability of foetal erythroblasts also severely limits the possibility of performing further analysis or studies on these cells.

To date, there is no antibody specific to primitive foetal nucleated red blood cell. There is thus a need to provide a novel marker of foetal nucleated red blood cells.

### SUMMARY OF THE INVENTION

The present invention is defined in the appended independent claims. Some optional features of the present invention are defined in the appended dependent claims.

The present invention is directed towards a method of detecting, separating and/or isolating at least one foetal nucleated red blood cell (FNRBC) from a sample comprising:
a) treating the sample using centrifugation in a density gradient medium comprising of a density selected from 1.077-1.120 g/ml;
b) contacting the sample with an antibody or antigen binding fragment thereof that specifically binds CD147 at a concentration of at least 20,000, thereby producing a mixture;
c) maintaining the mixture under conditions in which a complex forms between the agent and the CD147 in the sample; and
d) separating the complex from the mixture; thereby detecting, separating and/or isolating the FNRBC from the sample.

Shown herein is that CD147 may be a surface marker for detection, isolation and/or enrichment of foetal nucleated red blood cell (FNRBC) from a sample. The FNRBC can be further analyzed for prenatal disorders. Thus also provided herein are non-invasive methods for detecting FNRBCs and/or obtaining a prenatal diagnosis.

In other embodiments, the invention is directed to methods of detecting FNRBCs in a sample comprising contacting the sample with an antibody or antigen binding fragment thereof that specifically binds CD147, thereby producing a combination; maintaining the combination under conditions in which an immune complex forms between the antibody and FNRBCs present in the sample; and detecting whether the immune complex forms in; wherein if the immune complex is detected then FNRBCs are present in the sample.

In still other aspects, the invention further comprises methods of detecting prenatal disorders and/or the gender of a foetus.

### BRIEF DESCRIPTION OF THE DRAWINGS

FIG. 1A-D are images of differential immunocytochemical staining of CD147 on FNRBCs and adult anucleated red blood cells (AARBCs). (A) Negative control (primary antibody omitted) (B) Weak/undetectable immunostaining of anti-CD147 on AARBCs (C-D) Immunostaining of anti-CD147 on mixture of FNRBCs and AARBCs; intense staining on FNRBCs as opposed to that on AARBCs are seen.

FIG. 1E is a box plot showing mean intensity values (luminosity arbitrary units) for FNRBCs (n=450) and AARBC (n=358) as calculated using Adobe Photoshop Graphics Software and Luminosity Histogram, that were significantly different (p<0.001 Mann-Whitney test).

FIG. 2A is a bar graph showing the mean (%±SEM) recovery and purity of FNRBC with anti-CD147 antibody from model mixture using either Dynabead, MACS or FACS. ANOVA with Bonferroni correction, *P<0.01 and **P<0.001.

FIG. 2B are images of fluorescence-activated cell sorting (FACS) gating strategy for enrichment of FNRBCs from model mixtures tagged with anti-CD147 antibody conjugated to FITC. (i) Side scatter of AARBCs tagged with anti-CD147 antibody conjugated to FITC (control) (ii) Side scatter of model mixture (FNRBCs and AARBCs) tagged with anti-CD147 antibody conjugated to FITC. Both gated areas P2 (FITC-High/SSC-High) and P3 (FITC-High/SSC-Low) corresponds to target cell population FNRBCs. P1 gated area corresponds to AARBCs. P4 gated area corresponds to debris.

FIG. 3 are images of cytospun cells from enrichment of FNRBCs using anti-CD147 antibody from model mixtures with Dynabead, MACS and FACS separation techniques (A) Dynabead separation—positive fraction, (B) Dynabead separation—negative fraction (C) MACS separation—positive fraction (D) MACS separation—negative fraction (E) FACS separation—gated area P2 (F) FACS separation gated area P3. FNRBC (dotted arrow) and AARBC (solid arrow). Wright stain (200×). (Olympus BX61, Pennsylvania, USA).

FIG. 4 are images of immunocytochemical identification of e-globin positive FNRBCs from pre-TOP maternal blood (×400) (Olympus BX61, Pennsylvania, USA).

FIG. 5 are images of (A) FNRBCs from placental villi, Wright's stained (×400) (Olympus BX61, Pennsylvania, USA) (B) FNRBCs from placental villi unstained (C) FNRBCs from maternal blood with Hoechst stain (D) Maternal white blood cells Hoechst and CD45-AF488 stain. B-D Inverted microscope (×400) (Olympus IX70, USA).

FIG. 6 is a gel electrophoresis image of DNA product of whole genomic amplification. Sample 1-3: DNA product of whole genomic amplification (WGA) of 8-10 FNRBC manually picked from pre-TOP maternal blood (MB) and 8-10 FNRBC manually picked from placental villi (control) ran on 1% agarose gel electrophoresis.

FIG. 7 are Real-time PCR amplification curves of Beta-globin (HBB) and male sex determining region Y (SRY). Sample 1-3: Real-time PCR for Beta-globin (HBB) and male sex determining region Y (SRY) of maternal gDNA (control), foetal gDNA (control) and FNRBC isolated from pre-TOP maternal blood.

FIG. 8 is a graph showing the comparison on the recovery of cells using the different dilutions of anti-CD147 antibody in MACS. One way ANOVA with Bonferroni, p>0.05.

FIG. 9 are images of staining showing FNRBCs identified by morphological appearance after Wright's stain. Marker=20 μm

FIG. 10 are images of differential immunocytochemical staining Epsilon-globin positive FNRBCs (e+FNRBCs) after identification by Wright's stain, de-stained and immunocytochemistry performed. Marker=20 μm

FIG. 11 are graphs showing aCGH of epsilon-globin positive FNRBC (e+FNRBC) enriched from pre-TOP maternal blood. A: WGA amplified maternal genomic DNA obtained from maternal mononuclear cells shows profile female (46XX), control. B: Unamplified foetal genomic DNA (foetal trophoblast cells) shows profile male (46XY), control. C: 1 e+FNRBC from placental villi, laser microdissected (LCM) and WGA shows profile male (46XY), control. D: 1 e+FNRBC from maternal blood, laser microdissected (LCM) and WGA shows profile male (46XY). E: 3 e+FNRBC from maternal blood, laser microdissected (LCM) and WGA shows profile male (46XY). F: 5 e+FNRBC from maternal blood, laser microdissected (LCM) and WGA shows profile male (46X).

FIG. 12 is a schematic diagram showing the workflow of enriching FNRBCs from maternal blood, micromanipulation, and downstream molecular analyses. A: Maternal blood pre-TOP (10-20 ml; n=8); B: Density gradient centrifugation with Percoll™ 1.118; C: CD45-WBC depletion; D: CD147-positive selection of FNRBCs; E: Micromanipulator for manual picking of FNRBCs; F: FNRBCs stained Hoechst+/CD45 AF488- (White arrows; Marker=10 μm).

FIG. 13 are images of staining of FNRBCs for micromanipulation A: FNRBCs from placental villi, Wright's stained; B: FNRBCs from placental villi unstained; C: FNRBCs from maternal blood with Hoechst stain; D: Maternal white blood cells Hoechst and CD45-AF488 stain. Marker=10 μm.

FIG. 14 is a graph showing the DNA yield of 10-FNRBCs from villi and maternal blood after whole genome amplification as measured by picogreen. Mann Whitney, p>0.05; * Whiskers represent 95% confidence interval (CI).

FIG. 15 is a graph showing the DNA levels of WGA product of 10-FNRBCs and their fold increase (*) compared to theoretical genomic DNA in 10 cells.

FIG. 16 are a series of graphs showing the results of real time PCR of DNA amplified from 10-FNRBCs isolated from maternal blood with male and female pregnancies.

FIG. 17 are the results of 19 QF-PCR for amelogenin (AMXY) of male and female FNRBCs including DNA from their respective villi and maternal cells as controls.

FIG. 18 are results of real time and Quantitative fluorescent-PCR of FNRBCs from MB1 to MB8 and karyotype results. MB1 to MB4: FNRBCs from maternal blood of pregnancies with male foetus; MB5 to MB8: FNRBCs from maternal blood of pregnancies with female foetus.

FIG. 19 are images of fluorescence in situ hybridization (FISH) of FNRBCs isolated from maternal blood and villi samples. a and b: show male XY (arrow) signals in the FNRBCs isolated from maternal blood and villi respectively from a post-termination maternal blood sample; c and d: show female (XX) signal in the FNRBCs isolated from maternal blood and villi respectively from a post-termination maternal blood; e and f: show female (XX) signal in the FNRBCs isolated from maternal blood and villi respectively from a pre-termination maternal blood; g: FISH hybridization with LSI 13/21 probe, normal numbers of chromosome 13 (arrow) and 21 (arrow) in a FNRBC from post-termination maternal blood; h: control T21 cell-line showing 3 chromosome 21 signals after hybridization with LSI 13/21 probe.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

Reference to an element by the indefinite article “a” or “an” does not exclude the possibility that more than one of the element is present, unless the context clearly requires that there be one and only one of the elements. The indefinite article “a” or “an” as used herein thus usually means “at least one”.

The term “comprising” and its conjugations is used in its non-limiting sense to mean that items following the word are included, but items not specifically mentioned are not excluded. Accordingly, the term “comprising” encompasses the more restrictive terms “consisting essentially of” and “consisting of.”

The term “CD45 negative” as used herein refers to any cell that expresses no signal or is negative for native, recombinant or synthetic forms of the CD45 molecule/marker. The presence of CD45 expression on a cell in a sample may be determined using any immunostaining method known in the art and using any anti-CD45 reagent. Any cells positively stained with anti-CD45 reagent may be excluded as these may include CD45 positive white blood cells.

The term “erythroblast” as used herein refers to a red blood cell having a nucleus. In particular, an erythroblast refers to a nucleated precursor cell from which a reticulocyte develops into an erythrocyte. “Erythroblast” may be used interchangeably with a “Normoblast” and refers to a nucleated red blood cell, the immediate precursor of an erythrocyte. For example, the erythroblast may be of mammalian origin. In particular, the erythroblast may be a primitive or human foetal erythroblast. “Erythrocytes” or “red blood cells” or “RBC” include non-nucleated adult and foetal red blood cells. An example of an erythroblast may be foetal nucleated red blood cells (FNRBCs). These cells are the same or similar to foetal erythroblasts (hFEs).

The term “mammalian” is herein defined as a mammalian individual, in particular, a primate for example a human being. For purposes of research, the subject may be a non-human. For example the subject may be an animal suitable for use in an animal model, e.g., a pig, horse, mouse, rat, cow, dog, cat, cattle, non-human primate (e.g. chimpanzee) and the like.

The term “nucleated” as used herein refers to a cell that has a nucleus. Nucleated cells may be distinguished from red blood cells which are not nucleated based on any nuclear staining known in the art.

The term “prenatal disorder” as used herein refers to diseases or conditions in a foetus or embryo before it is born. The prenatal disorder may be selected from the group consisting of a chromosomal disorder, a genetic disorder, or a combination thereof. In particular, the prenatal disorder may be selected from the non-limiting group consisting of Down Syndrome, Edwards Syndrome, Patau Syndrome, a neural tube defect, spina bifida, cleft palate, Tay Sachs Disease, sickle-cell anemia, thalassemia, cystic fibrosis, fragile X syndrome, spinal muscular atrophy, myotonic dystrophy, Huntington's Disease, Charcot-Marie-Tooth disease, haemophilia, Duchenne muscular dystrophy, mitochondrial disorder, Hereditary multiple exostoses, osteogenesis imperfecta disorder, a combination thereof and the like.

The term “sample” as used herein refers to a subset of tissues, cells or component parts (for example fluids) that may include, but are not limited to, maternal tissue, maternal blood, cord blood, amniocenteses, chorionic villus sample, foetal blood, and/or foetal tissue/fluids. In particular, foetal tissue may be trophoblast tissue, placental tissue or a combination thereof. The sample as used in the present invention may have been previously subjected to a density gradient purification including, but not limited to, Ficoll gradient and Percoll™ gradient.

Epsilon-globin positive (e+) foetal nucleated red blood cells (FNRBCs) are ideal foetal cells in maternal blood for first trimester non-invasive prenatal diagnosis. Described herein is a surface antigen on e+FNRBCs that was identified by immunoscreening, and tested for suitability for separation of these cells in model mixtures and enrichment of e+FNRBCs from maternal blood. Most of the antigens tested were immunolocalised on both FNRBCs and adult anucleated red blood cells (AARBCs) except CD147, which had a statistically significant differential expression: stronger on the FNRBCs (122.46±22.21 AU) and weak/undetectable on AARBCs (205.11±7.08 AU) (P<0.05). From model mixtures, mean recovery of FNRBCs using MACS (79.7±0.7%) was significantly greater compared to Dynal-system (56.7±2.8%; P<0.01), FACS-P2 (FITC-High/SSC-High, 12.1±4.0%; P<0.001) and FACS-P3 (FITC-High/SSC-Low, 45.5±4.6%; P<0.001). Number of e+FNRBCs enriched from post-TOP maternal blood using anti-CD147 antibody was higher than with anti-GPA antibody (18 vs. 12 cells/15 ml); the number of slides for analysis were reduced by 55% in the former (p<0.05). Median recovery of first trimester e+FNRBCs per analysed slide was 300% higher using anti-CD147 antibody compared to anti-GPA antibody (0.53 vs. 0.18). Mean (±SEM) number of e+FNRBC enriched from pre-TOP maternal blood using anti-CD147 antibody is 20.3±2.8 cells (range: 6-46). FNRBCs may be isolated from pre-TOP maternal blood of 3 mothers carrying male foetuses were indeed male foetal cells by polymerase chain reaction (PCR) of the sex determining region Y (SRY) for foetal gender determination. Demonstrated herein is that CD147 may be strongly expressed on FNRBCs and is a marker for enrichment of FNRBCs from maternal blood for non-invasive prenatal diagnosis.

Two sources of foetal DNA may be available in the first trimester maternal blood, cell-free foetal DNA and foetal cells. The former may be limited to detecting paternally inherited disorders and trisomy 21, however, the latter may be more suitable for detecting single gene disorders. Of the foetal cells that enter the first trimester maternal circulation, foetal nucleated red blood cell (FNRBC) is the preferred target cell, because of its short life-span and hence it is unlikely to persist from a previous pregnancy, unlike the situation with foetal lymphocyte where this phenomenon could be the basis for a misdiagnosis. First-trimester FNRBC contain Epsilon-globin (e), an ideal foetal cell identifier which may be highly specific as expression declines after the first trimester.

At present, antibody targeting intracellular foetal hemoglobin (HbF) is being used to enrich FNRBCs. HbF however, may be elevated in the maternal AARBCs in some pregnancies. Embryonic hemoglobin ζ, though more specific, has a narrow temporal window of expression. Requirement of an additional permeabilization step to allow antibodies into the fragile FNRBCs causes rare cells to be lost during enrichment.

Cell surface antigens CD71, CD35, CD36, CD45, CD47 and GPA are most commonly used for enrichment of FNRBCs from maternal blood. CD71 is a transferrin receptor that is expressed on all cells that incorporate iron, such as activated lymphocytes, trophoblasts and erythroid cells from the burst forming units-erythroid (BFU-e) to the reticulocyte stage and definitive foetal NRBCs in maternal blood. While CD71 was strongly positive on 100% of foetal definitive erythroblasts and on 96% of maternal NRBCs, it was only expressed on ˜68% primitive foetal NRBCs in the first trimester. As such, use of CD71 to enrich first trimester FNRBCs may result in target cell loss. CD36 is not expressed on the FNRBCs, while CD35 and CD47 are expressed on both FNRBCs and AARBCs. GPA is present on all erythrocytes while CD45 is absent.

As described herein, intense and reproducible immunocytochemical staining of CD147 on the FNRBCs using immunoscreening for cell surface antigens and commercially available antibodies showed that surface antigen CD147 is a surface antigen that can be used for immunocellsorting of FNRBCs. CD147 is expressed on the erythrocyte lineage throughout erythroid development. Surface antigen CD147 showed statistically significant differential expression, stronger on the FNRBCs (122.46±22.21 AU) and weak/undetectable on AARBCs (205.11±7.08 AU). As shown herein, MACS with anti-CD147 was utilized as the main sorting technique as it showed most efficient recovery rates (79.7%) of FNRBCs from model mixtures compared to other techniques (FACS 12.1% and Dynal 56.7%).

Number of e+FNRBCs enriched from post-TOP maternal blood using anti-CD147 antibody was higher than with anti-GPA antibody (18 vs. 12 cells/15 ml); and number of slides for analysis were greatly reduced by 55% in the former (p<0.05). Median recovery of first trimester e+FNRBCs per analysed slide was 300% higher using anti-CD147 antibody compared to anti-GPA antibody (0.53 vs. 0.18).

Mean (±SEM) number of e+FNRBCs enriched from pre-TOP maternal blood using anti-CD147 antibody was 20.3±2.8 cells (range: 6-46 cells) and about 1.11 e+FNRBC per milliliter of maternal blood were enriched.

Foetal DNA of manually picked FNRBCs were amplified using whole genome amplification (WGA) technology which confirmed the enrichment of male FNRBCs using pre-TOP maternal blood of mothers carrying male foetuses (n=3).

Epsilon-globin positive (e+) foetal nucleated red blood cells (FNRBCs) may be considered the ideal foetal cells for first trimester non-invasive prenatal diagnosis. But, their rarity in maternal blood and lack of specific surface marker hampers their enrichment from the overwhelming background of adult anucleated red blood cells (AARBCs). Described herein is the selection of a surface antigen by immunocytochemical screening that separated FNRBCs and AARBCs allowing enrichment of FNRBCs from maternal blood for non-invasive prenatal diagnosis.

Accordingly, in one aspect of the present invention, there are provided methods of separating FNRBCs (e.g., foetal primitive NRBCs (FPNRBCs); foetal definitive NRBCs (FDNRBCs)) from a (one or more) sample comprising contacting the sample with an (one or more) agent that specifically recognizes CD147, thereby producing a mixture; maintaining the mixture under conditions in which a complex forms between the agent and the CD147 in the sample; and separating the complex from the mixture; thereby separating the FNRBCs from the sample.

The agent can be, for example, an antibody, a nanoparticle or a quantum dot. In a particular aspect, the agent is an antibody that specifically binds CD147. Examples of such antibodies include a polyclonal antibody, a monoclonal antibody, a chimeric antibody, a humanized antibody, portions thereof (e.g., antigenic fragment) or a combination thereof.

In specific embodiments, the FNRBCs are mammalian FNRBCs such as primate (e.g., human), canine, feline, equine, bovine, ovine, murine FNRBCs and the like.

In another aspect, there are provided a method of detecting, separating and/or isolating at least one foetal nucleated red blood cell (FNRBC) from a sample comprising:
a) treating the sample using centrifugation in a density gradient medium comprising of a density selected from 1.077-1.120 g/ml;
b) contacting the sample with an antibody or antigen binding fragment thereof that specifically binds CD147;
c) maintaining the mixture under conditions in which a complex forms between the agent and the CD147 in the sample; and
d) separating the complex from the mixture;
   thereby detecting, separating and/or isolating the FNRBC from the sample.

The concentration of the antibody or antigen binding fragment thereof may at least be 1:20,000 or 1:100. In particular, the concentration may be at least more than 1:10,000 or at least less than or equal to 1:100. The concentration of the antibody may be achieved by diluted the stock with at least one buffer, for example, phosphate buffered saline (PBS) and the like. The concentration of the antibody may be selected from the range of 1:10,000-1:100.

The method according to any aspect of the present invention may include the step of pre-treating the sample before step (b). In particular, treatment of the sample may include centrifugation of the sample in a density gradient medium. The density gradient medium for use in the present invention may comprise a colloid dispersed in a meltable gel. The colloid may impart the required density to the gradient medium. Thus, by altering the concentration of the colloid, the density of the medium may be correspondingly altered. The particulate nature of the colloid enables immobilization of separate layers of density without diffusion of one layer into another while in the gel state. Further, the colloid may be capable of maintaining the blood cells in a substantially unaggregated state. A non-limiting example of a colloid which imparts the density to the medium for use in any aspect of the method of the present invention may be polyvinyl-pyrrolidone coated silica, for example, Percoll™ manufactured by Pharmacia, and available from Sigma Chemical Co.

The density gradient medium for use in enriching foetal nucleated erythrocytes according to the invention is hypertonic. Under hypertonic conditions, red blood cells shrink and thus become more dense. Under these conditions, white blood cells maintain a constant density. Thus, by selectively shrinking the erythrocytes in a hypertonic medium, the density of these cells increases and they equilibrate within the gradient at a different density from the white blood cells.

The medium may be made hypertonic by the addition of salts to the centrifugation mixture. Suitable salts for use in the invention include sodium chloride, potassium chloride, or lithium chloride, or any mixture thereof. Commercially available balanced salt solution mixtures may also be used, such as Dulbecco's phosphate buffered saline (PBS), Hanks balanced salt solution, Earl's balanced salt solution and the like.

The Percoll™ solution may have a density gradient selected from the range 1.000-1.200 g/ml. In particular, the gradient may be selected from the range 1.050-1.150, 1.100-1120, 1.080-1.119, 1.078-1.119, 1.079-1.119, 1.100-1.119 g/ml and the like. In particular, the density gradient may be 1.118 g/ml.

The FNRBCs may be separated from anucleated red blood cells (ARBCs) present in a sample. If desired, the immune complex can be separated from the mixture using for example, immunomagnetic separation, flow cytometry or a combination thereof.

As will be appreciated by those of skill in the art, the FNRBCs separated and/or isolated using the methods described herein can undergo further testing. Examples of such testing includes fluorescent in situ hybridization (FISH), polymerase chain reaction (PCR), multiple ligand-dependent probe amplification (mpla), short tendon repeat analysis, array comparative genomic hybridization (CGH), genotyping, single plex sequencing, massively parallel sequencing and the like, or a combination thereof. In one aspect, the FNRBCs are further tested for sex discernment. In other aspects, the FNRBCs are further tested for one or more prenatal disorders. The one or more prenatal disorders include chromosomal disorders, genetic disorders (e.g., single gene disorder; multifactorial gene disorder), or a combination thereof. As will be appreciated by those of skill in the art such disorders can be found in the Online Mendelian Inheritance in Man (OMIM®) database (www.ncbi.nlm.nih.gov/omim). Specific examples of such disorders include Down Syndrome, Edwards Syndrome, Patau Syndrome, a neural tube defect, spina bifida, cleft palate, Tay Sachs Disease, sickle-cell anemia, thalassemia, cystic fibrosis, fragile X syndrome, spinal muscular atrophy, myotonic dystrophy, Huntington's Disease, Charcot-Marie-Tooth disease, haemophilia, Duchenne muscular dystrophy, mitochondrial disorder, Hereditary multiple exostoses, osteogenesis imperfecta disorder or a combination thereof.

In a particular aspect, the invention is directed to a method of determining a foetus' gender comprising contacting a maternal sample which comprises FNRBCs with an antibody or antigen binding fragment thereof that specifically binds CD147, thereby producing a mixture; maintaining the mixture under conditions in which an immune complex forms between the antibody or antigen binding fragment thereof and the CD147 present in the sample; separating the immune complex from the mixture, thereby separating FNRBCs from the sample; and testing the FNRBCs for the presence of the SRY gene, wherein if the SRY gene is present then the foetus is male and if the SRY gene is absent then the foetus is female.

In another aspect, the invention is directed to methods of detecting FNRBCs in a sample comprising contacting the sample with an antibody or antigen binding fragment thereof that specifically binds CD147, thereby producing a combination; maintaining the combination under conditions in which an immune complex forms between the antibody and FNRBCs present in the sample; and detecting whether the immune complex forms in the combination; wherein if the immune complex is detected then FNRBCs are present in the sample. In still other aspects, the invention further comprises methods of detecting prenatal disorders and/or the gender of a foetus.

The methods can further comprise one or more depletion steps in which the sample is further contacted with one or more agents (e.g., antibody) that removes (e.g., binds to) molecules other than FNRBCs. Thus, the deletion steps can be used to remove background noise such as cells other than FNRBCs. As will be appreciated by those of skill in the art, such molecules can then be detected by virtue of being recognized by the agent and removed from the sample. For example, the sample can be contacted with antibodies directed against CD45 and/or CD14 which will binds white blood cells (WBCs), thereby forming an immune complex and such immune complexes can be detected and/or removed from the sample. Other such molecules and methods for removing them are apparent to those of skill in the art.

In addition, the methods can further comprise contacting the sample with one or more agents that detect a nucleus in cell such as a nuclear staining agent. As will be appreciated by those of skill in the art, such agents include Hoescht stain, DAPI stain, and acridine orange.

Any suitable biological sample can be used in the methods and include a biological fluid (e.g., blood, cord blood) and tissue (e.g., trophoblast tissue, liver tissue, placenta). In particular aspects, the sample is a maternal sample (a sample obtained from a pregnant mother), and in other aspect, the sample is a foetal sample. In yet other aspects, the sample is obtained from a mother in a first trimester, a second trimester or a third trimester of a pregnancy.

A person skilled in the art will appreciate that the present invention may be practiced without undue experimentation according to the method given herein. The methods, techniques and chemicals are as described in the references given or from protocols in standard biotechnology and molecular biology text books.

### EXAMPLES

Standard molecular biology techniques known in the art and not specifically described were generally followed as described in Sambrook and Green, Molecular Cloning: A Laboratory Manual, Cold Springs Harbor Laboratory (Fourth Edition), New York (2012).

### Example 1

### Materials and Methods

Placental tissue and blood sample collection from women undergoing elective surgical termination of pregnancy (TOP) were approved by the Institutional Review Board. Blood samples were collected in EDTA coated vacutainers (Becton, Dickinson and Company, USA). FNRBCs were extracted from placental tissues following the protocol in Ponnusamy et al., 2008.

### Antigenic Profiling of FNRBCs Using Commercially Available Antibodies

Antibodies against selected surface antigens of AARBCs, other blood cells and their progenitors were tested for immunoreaction on both FNRBCs and AARBCs. Antibodies (raised in mouse, unless stated otherwise) against surface antigens CD34, CD46, CD55, CD100, CD175s, HLA-DR, CD117 (raised in rabbit) (Neomarkers, Lab Vision, California, USA); CD29, CD31, CD35, CD36, CD44, CD45, CD45RB, CD47, CD59, CD81, CD99, CD108, CD147, CD164, CD222, CD235a, E-Cadherin (BD Pharmingen, California, USA); CD14, CD90, CD105, CD133, Monocarboxylate transporter 1 (MCT1, raised in rabbit) (Chemicon, California, USA); and CD233 (Sigma-Aldrich, Missouri, USA); HLA-ABC (DAKO, California, USA) were used for immunocytochemical localisation of these antigens on the target cells following the protocol described in Ponnusamy et al., 2008. Briefly, cytospun slides were fixed in methanol/acetone (v/v, 1:1) for 2 min at room temperature, rinsed in PBS and air dried. After rehydration in PBST (1×PBS, Tween-20; Sigma, Missouri, USA), sequential incubations with 1) goat serum (Sigma) diluted 1:10 in PBS for 2 hours 2) anti-human murine or rabbit IgG-class antibody diluted 1:100 in PBS for 1 hour 3) biotinylated goat-anti-mouse antibody or goat-anti-rabbit antibody (Vector Laboratories, California, USA) for 1 hour 4) streptavidin conjugated with alkaline phosphatase (Vector Laboratories) for 30 min each were performed. All incubations were in humidifying chamber at room temperature. All washes between incubations were in PBST for 5 min. Freshly made Vector blue substrate (Vector Laboratories) was added onto the slides and incubated in the dark for 10 min. Slides were rinsed in distilled water for 30 sec; dehydrated in 100% ethanol for 30 sec and lastly air dried and observed under light microscope (Olympus BX61, Pennsylvania, USA).

### Measurement of Intensity of Immunostaining

The immunoreaction of various antibodies on the cell surfaces were scored for their staining intensities. In case of intense immunoreaction on FNRBCs, the staining intensities on AARBCs and FNRBCS were calculated, using luminosity histogram programme on graphics software Adobe Photoshop (Adobe Systems). Photographs of immuno-stained cells were digitized by reflective scanning at 300 dots per inch. Mean pixel intensity in positive cells (from up to five small squares of positive cells) (n=350-450 cells) was determined and luminosity (brightness) levels determined against a 256 grey scale (arbitrary units, AU). Data were analyzed for statistical significance of staining intensities (Choolani et al., 2003).

Model Mixtures of FNRBCs Spiked into AARBCs

Model mixtures used to test the enrichment efficiency of the protocols consisted of 1×10⁵ FNRBCs and 49×10⁵ AARBCs. AARBCs were prepared from peripheral venous blood collected in EDTA coated Vacutainers® (Becton, Dickinson and Company, USA). Blood sample was diluted using 1×PBS (1:1) and layered over equal volume of Ficoll-Paque (Sigma, Missouri, USA) and centrifuged (B Braun Biotech International B5, Pennsylvania, USA; 224×g, 30 min). An aliquot of RBC pellet was washed thrice using 1×PBS and counted using haemocytometer to prepare model mixtures. FNRBCs were isolated from placental tissue

Enrichment of FNRBCs from Model Mixtures

Three cell-sorting protocols were evaluated for the recovery and purity of FNRBCs enriched from model mixtures.

a. Dyna Beads Method:

FNRBCs were enriched from model mixtures (n=5) using Dynabead (Invitrogen Corporation, California, USA) separation system was carried out according to manufacturer's instructions with some modifications: Briefly, cells were suspended in 50 μl sorting buffer (sorting buffer: 0.5% BSA/PBS; 0.5M EDTA) and incubated with 1 μg anti-CD147 antibody (BD Pharmingen, California, USA) at 4PC for 30 min. Cells washed twice using 1×PBS were re-suspended in 500 μl sorting buffer and incubated with 5 μl pre-washed goat anti-mouse IgG conjugated Dynabeads M-450 (Invitrogen Corporation) at 4° C. for 30 min with slow end-over-end rotation. Magnetically bound cells were collected using Dynal Magnetic Particle Concentrator (MPC, Invitrogen Corporation). Unbound material was removed by aspiration and transferred into a new tube as negative fraction. Magnetically bound cells were washed twice and incubated with releasing buffer (Flow-Comp releasing buffer, Invitrogen Corporation) at room temperature for 15 min to free the cells from magnetic beads, and suspended in 500 μl sorting buffer. The tube was again placed in the MPC for 2 min and supernatant transferred into a new tube as positive fraction. Both positive and negative fractions were pelleted, re-suspended in 1×PBS for cell counting and subsequent cytocentrifugation onto slides (Shandon 3, Thermo Scientific, Massachusetts, USA). Slides were stained for further analysis.

b. Magnetically-Activated Cell Sorting (MACS):

Positive selection of FNRBCs from model mixtures (n=5) was performed through an indirect-magnetic labelling method of MACS Miltenyi Biotec, Bergisch Gladbach, Germany). Briefly, cells were incubated with anti-CD147 (1:10,000 in final incubation vol of 100 μl, BD Pharmingen) for 30 min at 4° C. and washed using MACS buffer (0.5% BSA/PBS; 0.5M EDTA; 500×g 10 min) and incubated with magnetic microbeads conjugated to rat anti-mouse IgG antibody (20 μL per 10⁷ cells in final incubation vol of 100 μl, Miltenyi Biotec.) for 30 min at 4° C. After incubation sample after washing was passed through Miltenyi miniMACS magnetic column. Positive fraction from the column was collected, washed and suspended for cell counting and subsequent cytocentrifugation onto slides. Slides were stained for further analysis.

c. Fluorescence-Activated Cell Sorting (FACS):

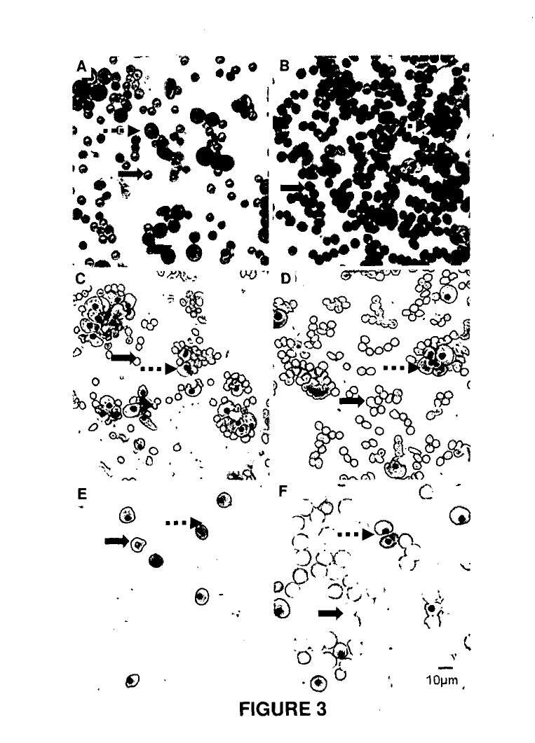

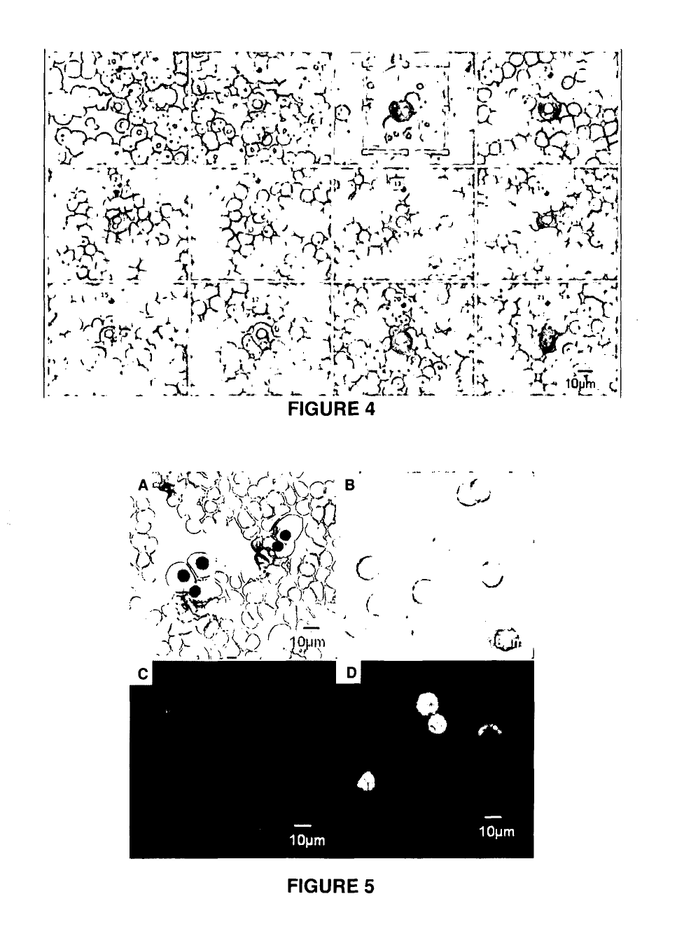

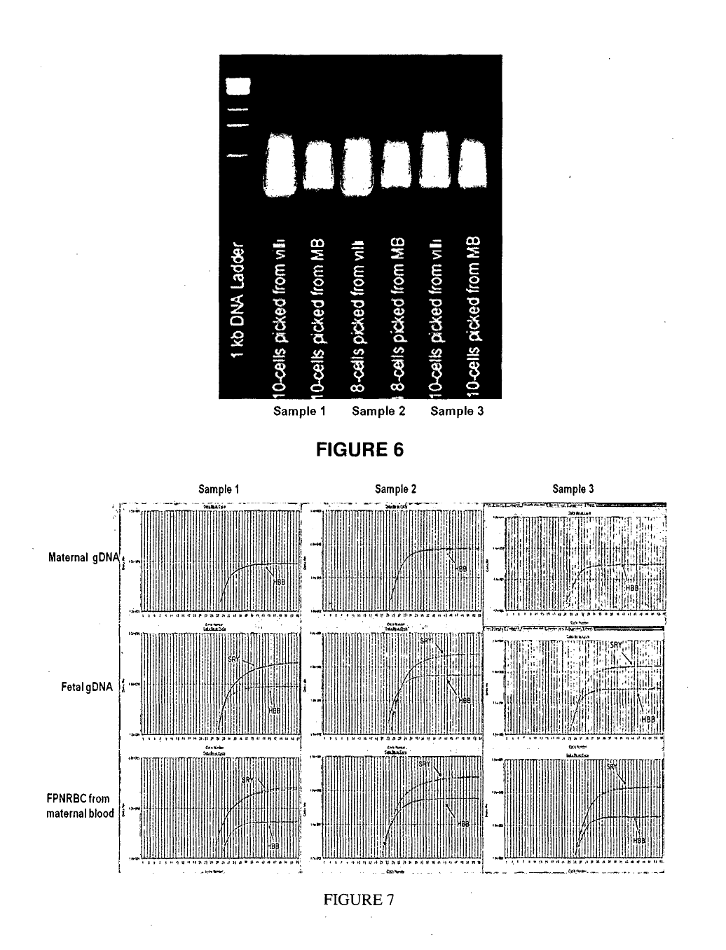

FACS to enrich FNRBCs from model mixtures (n=5) was performed using FACS Aria (Becton, Dickinson and Company, New Jersey, USA) according to manufacturer instructions with some modifications: Briefly, cells were incubated with FITC conjugated anti-human CD147 antibody (10 μl antibody/10⁶ cells; BD Pharmingen, USA) for 30 min at 4° C. in the dark with gentle tapping for every 5 min; washed in FACS buffer (0.5% BSA/PBS; 0.5M EDTA) and sorted. Isotype control staining was used to discard false-positive events. Cells were gated on forward (FITC-high or FITC-low) and side scatter (FITC-high/SCC-high or FITC-high/SCC-low) and sorting gates were established using pure AARBC samples as well as model mixtures both stained and unstained. Cells were sorted into four 15 ml Eppendorf (Eppendorf, Germany) tubes coated with 5% BSA (Sigma, Missouri, USA) and labelled P1 to P4 (P1-AARBC, P2-FITC-high/SCC-high, P3-FITC-high/SCC-low, P4-Debris) (FIG. 3). The cells were pelleted down, re-suspended into appropriate volume for cell counting and later cytospun onto slides to determine purity.

### Wright's Staining

Cytospun slides were air dried and Wright stained by sequentially immersing in 100% methanol containing additive (Aerofix Additive, Wescor, Utah, USA) (3 min), thiazin blue stain solution (Wescor, Utah, USA) (3 min), eosin stain solution (Wescor, Utah, USA) (3 min), rinsed in distilled water and air dried.

### Alkaline Phosphatase Immunocytochemistry for Identification of Epsilon-Globin Positive FNRBCs

Cytospun slides were Wright stained for morphological identification of FNRBCs. The locations of these cells were recorded. Slides containing FNRBCs were de-stained by placing them in 100%, 90% and 70% Ethanol for 5 min each. Epsilon globin positive FNRBCs were identified by alkaline phosphatase immunocytochemistry as per the protocol described in Ponnusamy et al., 2008.

Enrichment of FNRBCs from Maternal Blood to Compare the Efficiency of Anti-CD147 and Anti-Glycophorin A (Anti-GPA)

Briefly, 30 ml of peripheral venous blood was collected within 5 min of surgical procedure of termination of pregnancy (n=5) and divided into two equal parts to test two MACS protocols: enrichment of FNRBCs following negative depletion of CD45+ cells/GPA positive selection and negative depletion of CD45+ cells/CD147 positive selection of FNRBCs. Depletion of CD45+ white blood cells (WBCs) was similar for both protocols: 15 ml blood was diluted in PBS (1:1), layered over Percoll™ (ρ=1.118 g/ml; GE-Healthcare, Uppsala, Sweden) and centrifuged (3000 rpm, 30 min, brakes off). Cells in interface layer were collected, washed using 1×PBS containing 0.5% BSA and incubated with microbeads conjugated to anti-human CD45 (20 μl microbead/10⁷ cells; Miltenyi Biotec) for 30 min at 4° C. and washed to remove excess beads. Labelled cells were passed through LD-type MACS column (Miltenyi Biotec.). Cells in negative fractions were washed, counted and processed for selection of FNRBCs following either anti-GPA or anti-CD147 MACS procedures: For selection using anti-GPA, cells were incubated with magnetic beads conjugated to anti-GPA (20 μl microbead/10⁷ cells; Miltenyi Biotec.) for 30 min at 4° C. Incubated cells were washed and sorted using MS columns (Miltenyi Biotec.). Eluted positive fraction (i.e. CD45⁻/GPA⁺) was washed and cytospun onto glass slides. All washes were using MACS buffer (0.5% BSA/PBS; 0.5M EDTA) for centrifugation (500×g) for 10 min.

For selection using anti-CD147, cells from negative fraction were subjected to two successive incubations (each 30 min at 4° C., gentle tapping for every 6 min) and washings in between before sorting were carried out: first incubation with anti-CD147 antibody (stock antibody diluted 1:1000 in 1×PBS; from the diluted antibody 10 μl added per 10⁷ cells; in final incubation vol of 100 μl, BD Pharmingen) and the second with magnetic microbeads conjugated to rat anti-mouse IgG antibody (40 μL per 10⁷ cells in final incubation vol of 100 μl, Miltenyi Biotec.) Eluted positive fraction (i.e. CD45⁻/CD147⁺) was washed and cytospun onto slides. FNRBCs were morphologically identified by Wright's staining and confirmed its identity by ε-globin immunocytochemistry.

Enrichment of FNRBCs from Pre-TOP Maternal Blood

Two sets of maternal blood samples were collected before surgical procedure (pre-TOP maternal blood samples): First set of samples, (each 10-20 ml; n=14) were processed following negative depletion of CD45+ cells/CD147 positive selection of FNRBCs described elsewhere in this paper to calculate the number of immunostained e+FNRBCs on slides from each sample. The second set of samples (each 20 ml; n=3) were processed identically to enrich FNRBCs which were not cytospun but stained with anti-CD45-AF488 and Hoechst dyes for manual cell picking.

### Staining of Cells for Manual Cell Picking

CD147+ cells enriched from maternal blood (containing FNRBCs and AARBCs) were re-suspended in 1 ml of culture medium and incubated with 5 μl of anti-CD45 conjugated to fluorophore AF488 (Invitrogen, USA) for 1 hour, and a DNA labelling Hoechst stain (1 μl stock/1 ml cell suspension, stock conc. 10 mg/ml; Invitrogen, USA) was added and incubated for 15 min. Cells were spun down at 3000 rpm for 5 min, and re-suspended in culture medium. Stained cells were examined under fluorescent microscope (Olympus IX70, USA). All incubations were performed at 37° C. Human peripheral blood mononuclear cells served as controls for staining, which are both CD45 and Hoechst positive.

### Manual Picking of Hoechst Stained Cells Using Micromanipulator

Individual FNRBCs which are Hoechst positive and CD45-AF488 negative were manually picked with the help of a micromanipulator (Narishige, Japan) and an inverted microscope (Olympus IX70, USA). Stained cells were re-suspended in culture medium (1-3×10⁶ cells/100 μl of medium). In a 60 mm culture dish, 100 μl of anti-CD45-AF488/Hoechst stained cell suspension, 50 μl of culture medium (for collection of picked cells) and 50 μl of 1×PBS (for washing of cells) were loaded as separate droplets. A micropipette (Origio, USA) with an internal diameter of 20 μm was placed in the stained cell suspension droplet. Hoechst positive and CD45-AF488 negative FNRBCs were identified and manually picked and serially transferred to the droplets containing medium and 1×PBS before being transferred to PCR tubes and stored at −20° C. for further analysis. The culture medium is made of Iscove's modified Dulbecco's medium (IMDM, Invitrogen Gibco, USA)+30% foetal bovine serum (FBS, Biochrom AG, Germany)+1% bovine serum albumin (BSA, Invitrogen Gibco, USA)+10⁻⁴ mol/β-mercaptoethanol (Invitrogen Gibco, USA)+100 μg/ml iron-saturated transferrin (Sigma-Aldrich, USA)+1% antibiotic antimycotic (Invitrogen Gibco, USA).

### Whole Genome Amplification (WGA)

Manually picked cells were lysed using 7 μl Cell Extraction Buffer from the Picoplex™ WGA kit (Rubicon Genomics, MI, USA). DNA was extracted by 75° C. incubation for 10 min followed by 95° C. incubation for 4 min using the Extraction Cocktail provided in the kit. WGA was performed using the Picoplex™ WGA kit (Rubicon Genomics) according to manufacturer's recommendations with 16 cycles of amplification. The quality and quantity of all amplified DNA samples were assessed by gel electrophoresis before performing PCR. DNA from CD45+ cells from mother and placental cells were also extracted.

### Real-Time PCR for Foetal Sex Determination

Real-time PCR analysis was done using PE Applied Biosystems 7000 Sequence Detector (Applied Biosystems, CA, US). Beta-globin (HBB) an endogenous control and a male sex determining region Y (SRY) foetal gender determination were analysed. The following SRY and HBB primers and probes (AIT Biotech, Singapore) were used: SRY-forward, 5′-TGG CGA TTA AGT CAA ATT CGC-3′ (SEQ ID NO:1); SRY-reverse, 5′-CCC CCT AGT ACC CTG ACA ATG TAT T-3 (SEQ ID NO:2)′; SRY-probe, 5′-(6-FAM) AGC AGT AGA GCA GTC AGG GAG GCA GA (TAMRA) (SEQ ID NO:3); HBB-forward, 5′-GTG CAC CTG ACT CCT GAG GAG A-3′ (SEQ ID NO:4); HBB-reverse, 5′-CCT TGA TAC CAA CCT GCC CAG-3′(SEQ ID NO:5); HBB-probe, 5′-(VIC) AAG GTG AAC GTG GAT GAA GTT GGT GG (TAMRA)-3′(SEQ ID NO:6). Commercial male genomic DNA (Promega, Madison, USA) with known initial concentrations was serially diluted (5-folds) to generate the standard curves for HBB and SRY. Samples and standards were run in triplicates in the same assay. Water blanks were included in triplicates for each PCR as amplification negative controls. Reactions were set up in a reaction volume of 25 μl using the TaqMan Universal PCR Master Mix (Applied Biosystems). SRY and HBB primers were used in a final concentration of 450 nM and probes at a final concentration of 225 nM respectively. Three microliters of WGA product and 60 ng of genomic DNA were used for amplification. Thermal cycling for both SRY and HBB was initiated with a 2-min incubation at 50° C., to allow the uracil N-glycosylase (UNG) to act, followed by a first denaturation step of 10 min at 95° C. and then 55 cycles of 95° C. for 15 s and 60° C. for 1 min.

### Statistics

Statistical differences were determined by ANOVA with Bonferroni post-correction, Wilcoxon signed rank test or Mann-Whitney test using SPSS statistical software (SPSS Inc. USA) and GraphPad (GraphPad Software Inc. USA).

### Results

FNRBCs were extracted from placental villi obtained from patients undergoing termination of pregnancy (TOP). Immunolocalization of thirty-one selected surface antigens on FNRBCs and AARBCs was studied by immunocytochemistry (ICC): CD147 (Basigin), showed differential expression between the cell types. Anti-CD147 antibody was used to enrich FNRBCs from model mixtures (FNRBC 1×10⁵ cells: AARBC 49×10⁵ cells; n=5) using immunomagnetic (magnetically-activated cell sorting (MACS) and Dynal system) and flow cytometric (FACS) separation methods to assess the efficiency of recovery. FNRBCs from 15 ml post-TOP maternal blood (n=5) were enriched following DGC/MACS using anti-CD45 and anti-CD147 antibodies; and this protocol was also compared using anti-GPA magnetic beads in place of anti-CD147. Enriched FNRBCs were identified by both morphological and immunological (e-globin) methods. Recovery of e+FNRBCs from maternal blood was calculated. FNRBC from 20 ml pre-TOP maternal blood (n=14) were enriched using anti-CD147 and identified by both morphological and immunological (e-globin) method. As proof-of-principle, 20 ml pre-TOP maternal blood (n=3) was obtained from mothers with male pregnancies and, as shown herein by polymerase chain reaction (PCR) of the SRY gene, the enriched FNRBCs were indeed foetal cells.

Immunocytochemical screening showed that surface antigen CD147 had statistically significant differential expression, stronger on the FNRBCs (122.46±22.21 AU) and weak/undetectable on AARBCs (205.11±7.08 AU). MACS recovered 79.7% FNRBCs from model mixtures compared to other techniques (FACS 12.1% and Dynal 56.7%) and purity of separated mixtures was the highest with FACS (64.9%) compared to other two techniques (13%). Number of e+FNRBCs enriched from post-TOP maternal blood using anti-CD147 antibody was higher than with anti-GPA antibody (16 vs. 12 cells/15 ml); and number of slides for analysis were greatly reduced by 55% in the former (p<0.05). Median recovery of first trimester e+FNRBCs per analysed slide was 300% higher using anti-CD147 antibody compared to anti-GPA antibody (0.53 vs. 0.18). Median (±SD) number of e+FNRBCs enriched from pre-TOP maternal blood using anti-CD147 antibody was 20±3 cells (range: 6-46 cells) and about 1.16 e+FNRBC per milliliter of maternal blood were enriched. Foetal DNA from FNRBCs was amplified using single cell whole genome amplification (WGA) technology and the enrichment of male FNRBC from pre-TOP maternal blood of mothers carrying male foetuses (n=3) was confirmed.

### Conclusion

Despite the accuracy of current invasive foetal testing, the incidence of Down's syndrome remains approximately 1 in 1000 live births. This is because the invasive tests are reserved for a minority of pregnancies, pregnant woman with advanced maternal age (>35 years of age). Even though 4/5 of babies with Down's syndrome are born to mothers under the age of 35 years, they are not offered invasive testing because the risk of iatrogenic complication is greater than the incidence of Down's syndrome in the given foetus. Non-invasive prenatal diagnosis (NIPD) could eliminate the need for invasive foetal testing.

Isolating and analyzing foetal DNA from as little as 1 FNRBC recovered from amongst a million nucleated maternal cells is possible with the use of automated micromanipulation, laser capture microscopy systems and downstream analysis of foetal cell with single cell whole genomic amplification coupled with array CGH technologies. Therefore, it is not inconceivable that very small numbers of foetal cells (˜20 cells) enriched from maternal blood from an ongoing euploid pregnancy is likely sufficient for noninvasive prenatal diagnosis.

Current protocol for isolation of FNRBC is time-consuming, labour-intensive and co-isolation of large numbers of non-nucleated adult red blood cells results in lower purity. These limitations can be overcome by automation of various steps of the protocol.

Invariably FNRBCs are lost during multiple steps of the enrichment protocol especially at the very first step, density gradient centrifugation. Separation of FNRBCs from large background of maternal AARBCs is possible with microfluidic separation devices. Such separation systems have been examined, in collaboration with Institute of Bioengineering and Nanotechnology Singapore. The use of microfluidic device was developed to separate the two cell types, FNRBCs and AARBCs based upon their physical properties such as size and deformability. The silicon-based cross-flow microfilter device recovered 74% of FNRBC and depleted half the AARBCs at a flow rate of 0.3 mL/min from model mixtures. This suggests that 20 mL of maternal blood could be processed in just over an hour. However, recovery efficiency of these microfluidic devices can be further enhanced by combining nano-patterned surfaces with subsequent coating of the specific cell adhesion molecules. New microfluidic designs coated with specific cell adhesion molecules on the surface showed an enhanced separation time and ratio of captured cells.

Manual slide scanning for FNRBC depends heavily on the observer. Duration taken to scan slides depends on the efficiency of the observer and is subject to observer error. Automation of this process is possible with platforms such as MetaFer (MetaSystems, Altlussheim, Germany), IMSTAR Pathfinder (Paris, France) and Ikoniscope (Ikonysis, Connecticut, USA). For example MetaFer system enables automated imaging by morphology that combines markers and fluorochromes. The MetaFer system platform is highly throughput as it performs fast scanning (6-15 mins/slide) using a high resolution CCD camera and automated slide loader. The automated slide loader has the capacity to read 80 slides which can load up to ten 8-slide frames to the scanning stage automatically and unattended.

It is possible to screen and analyse FNRBC obtained from maternal blood using an automated platform consisting of a commercially available colony picking instrument coupled with to customized software. Choi has demonstrated the utility of this automated approach to identify and isolate target cells and accomplished 100% selectivity and specificity. Long and colleagues, designed and tested an effective algorithm for cell recognition in brightfield microscopy which frees users from using costly fluorescence probes and limited number of available fluorescence channels.

Huang et al (2008) enriched FNRBCs from the peripheral blood of 58 pregnant women using a microfluidic system that combined a microfluidic chip for size-based cell separation (CSM) and a magnetic device for hemoglobin-based cell isolation (HE). The microfluidic system was able to enrich a mean of 37.44 FNRBC per milliliter of maternal blood (range 0.37-274.36 FNRBC/mL). CSM/HE system is able to process 5 to 20 milliliter of maternal whole blood in 2 to 6 hours.

Kilpatrick et al (2004) and Seppo et al (2008) utilized the Ikoniscope robotic microscopy platform specifically for foetal cell identification and analysis. The detection of foetal cells in maternal circulation was investigated using two fundamental approaches; FISH-based scanning and antibody-based scanning. In the former, foetal cells were identified based on the presence of a Y-chromosome in male-foetus pregnancies and latter foetal cells were identified based on the expression of foetal haemoglobin. Foetal cells were identified in 27 of the 29 samples from male pregnancies. Up to 175 slides are fed to the stage by an automated slide/cassette feeder allowing unattended processing and operating on a 24-hours/7-days a week basis.

Protein glycosylation on the surface of FNRBCs and AARBCs was suggested to be different. Such a difference was used for cell separation by differential lectin binding selection (soyabean agglutinin, SBA). The usefulness of SBA for the enrichment of foetal NRBCs was demonstrated by Kitagawa et al. (2004) whereby, a mean of 6.57+/−7.12 cells were recovered from 1 mL of maternal blood. In a similar study, Shinya et al. (2004) identified foetal gene in all seven cases using SBA isolation, micromanipulation and PCR analysis.

Demonstrated herein is that CD147 is strongly expressed on primitive FNRBCs and is a marker for the enrichment of FNRBCs from maternal blood for non-invasive prenatal diagnosis. Accordingly, provided herein are improved methods for obtaining, separating and/or isolating FNRBCs.

TABLE 1
Mean intensity classification based on immunocytochemical
staining of 31 antigens on FNRBCs and AARBCs.

| | Mean Intensity | | |
|---|---|---|---|
| | Classification* | | |
| Group | FNRBCs | AARBCs | Target Antigens |
| A | ++ | + | CD147 |
| B | ± | − | CD164, MCT1 |
| C | + | + | CD44, CD55, CD59 |
| D | ++ | ++ | CD47, CD233, CD235a |
| E | − | − | CD14, CD29, CD31, CD34, CD35, |
| | | | CD36, CD45, CD45RB, CD46, |
| | | | CD81, CD90, CD99, CD100, |
| | | | CD105, CD108, CD117, CD133, |
| | | | CD175s, CD222, E-caderin, |
| | | | HLA-ABC, HLA-DR |

*All representative microphotographs of immunostained FNRBCs and AARBCs were converted to 256 grey scale (0 = black and 256 = white). Intensity value (arbitrary units, AU) was calculated using Adobe Photoshop Graphics Software and Luminosity Histogram. Mean intensity value of 187.3 AU (range: 185.2-189.2 AU, n = 5) was used as a cut off representing background staining. Mean intensity of FNRBCs and AARCs for each antigen was recorded and classified: mean value of ≧187.3 AU was regarded as not stained (−), 187.2-109.9 AU as low intensity stained (+), ≦110.0 AU as high intensity stained (++) and partial staining of cells (±).

TABLE 2
Comparison of enrichment of e+FNRBCs from post-TOP
maternal blood using anti-CD147 and anti-GPA in MACS.

| | | Median number | Median number | Number of e− |
|---|---|---|---|---|
| | | of e+FNRBCs | of slides | FNRBC per |
| | N | (Range) | (Range)^{a} | slide |
| Anti-CD147 | 5 | 16 (7-32) | 30 (9-46) | 0.53 |
| Anti-GPA | 5 | 12 (10-86) | 67 (27-93) | 0.18 |

^{a}Wilcox signed rank test P < 0.05

TABLE 3
Enrichment of FNRBCs from pre-TOP maternal blood (n = 15)

| | |
|---|---|
| Maternal age | Mean (±SEM): 28.4 ± 2.3 years |
| | (range: 18-48 years) |
| Gestational age of foetus at TOP | Mean (±SEM): 8.9 ± 0.2 weeks |
| Total volume of blood processed | 275 ml (mean 18.3 ml, median 20 ml) |
| Total e+FNRBC enriched | 304 (range: 6-46 cells/sample |
| e+FNRBC/sample | Median (±SD): 20.3 ± 2.8 |
| e+FNRBC/ml | 1.11 cells/ml |

### Example 2

### Samples

### Ethical Approval for Use of Human Samples

Collection of human tissue and blood samples for this study was approved by the Institutional Review Board, National University Hospital, National Healthcare Group, Singapore. All research participants gave written informed consent for the collection and use of human samples.

### First Trimester Placental Tissue

First trimester placental tissues (7+6 to 10+4 weeks) were collected from women undergoing elective (for social reasons) surgical termination of pregnancy (TOP). Gestational age was determined by ultrasound measurements of the crown rump length of the foetus. Placental tissue was collected from within the products of conception and placed directly into sterile containers containing PBS solution. Separately, a small portion of placental tissue was sent for karyotyping.

Peripheral Blood from Pregnant Women Undergoing Elective TOP
Pre-TOP blood samples

Peripheral blood samples (10-20 ml) were collected from women undergoing elective TOP before surgical procedure in EDTA coated vacutainers (Becton, Dickinson and Company, USA).
Post-TOP blood samples

Peripheral blood samples (10-30 ml) were collected from women undergoing elective TOP within five minutes of completion of surgical procedure in EDTA coated vacutainers (Becton, Dickinson and Company, USA).
Peripheral blood from non-pregnant volunteers

Peripheral blood samples (20 ml) were collected from healthy male and non-pregnant female volunteers in EDTA coated vacutainers (Becton, Dickinson and Company, USA).

### Methods

Isolation of FNRBCs from Placental Tissue

Placental villi was carefully dissected off adjacent deciduas and washed twice in PBS (First BASE, Singapore) to remove maternal blood contamination. The cleaned villi were minced and incubated in 45 ml of trophoblast digestion buffer containing trypsin (Gibco Invitrogen Corporation, California, USA), HBSS (Gibco Invitrogen), 1M HEPES (Gibco Invitrogen)) at 37° C. for 20 min with moderate shaking. Trypsin-activity was stopped with the addition of 5 ml foetal calf serum (Sigma-Aldrich, Missouri, USA) and strained through a 70 μm cell strainer (Becton, Dickinson and Company, New Jersey, USA) to obtain a single cell suspension which was then centrifuged at 2095×g (Beckman Allegra X-15R, Beckman Coulter, California, USA) at room temperature for 10 min. The cell pellet was re-suspended in 5 ml of PBS and layered over Percoll™ 1083 (GE Healthcare Bio-sciences, Uppsala, Sweden) and centrifuged at 2095×g for 20 min with brakes off. The pellet containing FNRBCs was washed twice with PBS and re-suspended.

### Cell Count

Cell sample is diluted with PBS and 10 μl is loaded onto the Neubauer haemocytometer (Sigma-Aldrich). Cells are counted under a microscope (Olympus BX61, Pennsylvania, USA). Four separate areas were examined and the average number of cells, N, per 1 mm2 (0.1 μl volume) was determined and the concentration of cells in the original sample calculated (N×Dilution×10⁴ per ml).

### Preparation of Cytospun Slides

Up to 5×10⁴ cells were suspended in 300 μl 0.5% BSA/PBS (w/v) loaded into a cytospin chamber and centrifuged onto a slide at 500 rpm for 5 min inside the cytocentrifuge (Thermo Scientific Shandon 4 Cytospin, Leicestershire, England). Slides were air-dried and processed immediately or stored in foil and Parafilm at −20° C. FNRBCs enriched from maternal blood using anti-CD147 antibody and micromanipulated onto glass slides were centrifuged at 700 rpm for 5 min.

### Identification of FNRBCs by Morphology and Immunocytochemistry

Wright's staining

Cytospun slides were air-dried and Wright stained by sequentially immersing in 100% methanol containing additive (Aerofix Additive, Wescor, Utah, USA) (3 min), eosin stain solution (Wescor) (3 min), thiazin stain solution (Wescor) (3 min), rinsed in distilled water and air-dried.
Identification of foetal nucleated red blood cells (FNRBCs)

Cytospun slides were Wright stained for morphological identification of FNRBCs. The locations of these cells were recorded. Slides containing FNRBCs were de-stained by placing them in 100%, 90% and 70% Ethanol for 5 min each. Epsilon-globin-positive FNRBCs (e+FNRBCs) were confirmed by alkaline phosphatase immunocytochemistry. Slides were fixed in methanol/acetone (v/v, 1:1) for 2 min at room temperature, rinsed in PBS and air-dried. After rehydration in PBST (1×PBS, Tween-20; Sigma), sequential incubations with: 1) goat serum (Sigma) diluted 1:10 in PBS for 2 hours; 2) mouse IgG anti-human epsilon-globin antibody (Fitzgerald Industries, Massachusetts, USA) diluted 1:100 in PBS overnight at 4° C.; 3) biotinylated goat-anti-mouse antibody (Vector Laboratories) for 1 hour; 4) streptavidin conjugated with alkaline phosphatase (Vector Laboratories) for 30 min were performed. All incubations were in humidifying chamber at room temperature. All washes between incubations were in PBST for 5 min. Freshly made Vector blue substrate (Vector Laboratories) was added onto the slides and incubated in the dark for 10 min. Slides were rinsed in distilled water for 30 sec; dehydrated in 100% ethanol for 30 sec and lastly air dried. The slides were observed under light microscopy (Olympus BX61).

### Density Gradient Centrifugation

Preparation of Percoll™ 1118

Percoll™ (GE Pharmacia) is purchased commercially as a density gradient of 1.130 g/ml. Percoll™ gradients 1.083 and 1.118 were prepared from this stock solution by dilution with 1.5M NaCl (p=1.058 g/ml) (Percoll™ Methodology and Applications 2nd Edition, Amersham Pharmacia Biotech, UK). Percoll™ gradients were prepared according to formula

$$V 0 = V \frac{ρ - 0.1 {ρ}^{′} - 0.9}{ρ0 - 1} ,$$

where
V0=volume of Percoll™ stock (1.130 g/ml)
V=volume of the final working solution in ml
ρ=desired density of final working solution in g/ml
ρ0=density of Percoll™ stock in g/ml (1.130 g/ml)
ρ′=density of 1.5M NaCl (1.058 g/ml)
1.5M NaCl to make up to final working solution volume
Measurement of Percoll™ 1118 density

Measurement of density was performed using a Densito 30PX densitometer (Mettler Toledo, Ohio, USA) and 1.118 g/ml gradient achieved by adding 1.5M NaCl.
Centrifugation

Maternal blood is diluted 1:3 in PBS and gently layered over an equal volume of Percoll™ 1118 (maternal blood:PBS:density gradient 1:3:1) in 15 ml Falcon tubes (BD Biosciences). Samples were centrifuged at 2095×g (Beckman Coulter) for 30 min at 20° C. with brakes off. Cells from the interface layer were collected.

### Titration of Anti-CD147 Antibody Concentration for MACS

To determine the optimal anti-CD147 antibody concentration for the separation of FNRBCs from model mixture, model mixtures of FNRBCs and adult RBCs were prepared (1×10⁵ FNRBCs spiked into 49×10⁵ adult RBCs). Titration of anti-CD147 antibody was performed with dilutions of antibody in PBS—1:100, 1:1,000, 1:10,000 and 1:20,000. Model mixtures (n=3) were incubated successively with different antibody preparations and secondary antibodies tagged with magnetic beads and separated by MACS system. Cells collected from the positive fraction (CD147+) and negative fraction (CD147−) were counted using haemocytometer and cytospun onto slides.

Enrichment of Epsilon-Globin Positive FNRBCs (e+FNRBCs) from Pre-Termination of Pregnancy Maternal Blood

Pre-TOP maternal blood was collected (10-20 ml; n=20), then processed following negative depletion of CD45+ cells and CD147 positive selection of FNRBCs. Briefly, 20 ml blood was diluted in PBS (1:3), layered over Percoll™ 1118 (GE-Healthcare) and centrifuged Cells in interface layer were collected, washed using 1×PBS containing 0.5% BSA and incubated with microbeads conjugated to anti-human CD45 (20 μl microbead/10⁷ cells; Miltenyi) for 30 min at 4° C. and washed to remove excess beads. Labelled cells were passed through LD-type MACS column (Miltenyi). Cells in negative fractions were washed, counted and subjected to two successive incubations (each 30 min at 4° C., gentle tapping for every 6 min) and washings in between before sorting was carried out: first incubation with anti-CD147 antibody (stock antibody diluted 1:1000 in 1×PBS; from the diluted antibody 10 μl added per 107 cells; in final incubation volume of 100 μl, BD Pharmingen) and the second with magnetic microbeads conjugated to rat anti-mouse IgG antibody (40 μl per 10⁷ cells in final incubation volume of 100 μl, Miltenyi). Eluted positive fraction (i.e. CD45−/CD147+) was washed and cytospun onto slides. FNRBCs were morphologically identified by Wright's staining and its identity confirmed by epsilon-globin immunocytochemistry. Cells were cytospun onto glass slides. Slides were Wright stained and observed morphologically. Slides with FNRBCs were identified and de-stained for epsilon-globin immunocytochemistry.

Enrichment of FNRBCs from Pre-Termination of Pregnancy Maternal Blood and Micromanipulator Assisted Manual Cell Picking of FNRBCs

Pre-TOP maternal blood was collected (10-20 ml; n=8) and were processed following negative depletion of CD45+ cells and CD147 positive selection of FNRBCs described above to enrich FNRBCs for micromanipulator assisted manual cell picking. Cells from CD147+ fraction were stained as described and micromanipulator assisted manual picking of FNRBCs performed.

Fluorescent Staining of CD147+ Cells Enriched from Maternal Blood for Micromanipulator Assisted Manual Cell Picking

CD147+ cells enriched from maternal blood (containing FNRBCs and adult RBCs) were re-suspended in 1 ml of culture medium¹ and incubated with 5 μl of anti-CD45 conjugated to fluorophore AF488 (Invitrogen) for 1 hour, and a DNA labelling Hoechst stain (1 μl stock/1 ml cell suspension, stock conc. 10 mg/ml; Invitrogen) was added and incubated for 30 min. Cells were spun down at 3000 rpm for 5 min, and re-suspended in culture medium. Stained cells were examined under fluorescent microscope. (Olympus IX70). All incubations were performed at 37° C. Human peripheral blood mononuclear cells served as controls for staining, which are both CD45-AF488 and Hoechst positive. ¹Culture medium: Iscove's modified Dulbecco's medium (Invitrogen Gibco)+30% foetal bovine serum (Biochrom AG, Germany)+1% bovine serum albumin (Invitrogen Gibco)+10⁻⁴ mol/l β-mercaptoethanol (Invitrogen Gibco)+100 μg/ml iron-saturated transferrin (Sigma-Aldrich)+1% antibiotic antimycotic (Invitrogen Gibco).

### Manual Picking of Hoechst Stained Cells Using Micromanipulator

Individual FNRBCs which are Hoechst positive and CD45-AF488 negative were picked using a micromanipulator (Narishige, Japan) and an inverted microscope (Olympus IX70). Stained cells were re-suspended in culture medium (1-3×10⁶ cells/100 μl of medium). In a 60 mm culture dish, 100 μl of CD45-AF488/Hoechst stained cell suspension, 50 μl of culture medium (for collection of picked cells) and 50 μl of 1×PBS (for washing of cells) were loaded as separate droplets. A micropipette (Origio, USA) with an internal diameter of 20 μm was placed in the stained cell suspension droplet. Hoechst positive and CD45-AF488 negative FNRBCs were identified and manually picked and serially transferred to the droplets containing medium and 1×PBS before being transferred to PCR tubes and stored at −20° C. for whole genome amplification.

### Whole Genome Amplification (WGA)

Manually picked cells were lysed using 3 μl Cell Extraction Buffer from the Sureplex™ WGA kit (BlueGnome, Cambridge, England). DNA was extracted by 75° C. incubation for 10 min followed by 95° C. incubation for 4 min using the Extraction Cocktail provided in the kit. WGA was performed using the Sureplex™ WGA kit (BlueGnome) according to manufacturer's recommendations with 16 cycles of amplification. The quality and quantity of all amplified DNA samples were assessed by PicoGreen® assay before performing PCR.

PicoGreen® Assay Measuring DNA Yield after Whole Genome Amplification

A DNA standard curve was prepared using 2 μg/mL stock standard (Promega, Wisconsin, USA) in TE. Five-point standard curve from 1 μg/mL to 1 μg/mL is prepared according to dilutions below in Table 4. PicoGreen® (Promega) is added to the working solution in a 96-well microplate, thoroughly mixed and incubated for 5 minutes at room temperature, protected from light. Sample DNA is diluted in TE to a final volume of 100 μl each in a 96-well microplate. 100 μl PicoGreen® is added to the sample wells. Mixture is incubated 5 minutes in the dark and the fluorescence measured with the DTX 800/880 Multimode Detector (Beckman Coulter).

TABLE 4
Dilutions prepared for the five-point standard curve
from 1 ng/mL to 1 μg/mL.

| Volume (μl) of | | | |
|---|---|---|---|
| 2 μg/ml lambda | | | Final DNA |
| bacteriophage | Volume | Volume of diluted | concentration in |
| stock | of TE (μl) | PicoGreen ® (μl) | PicoGreen ® assay |
| 100 | 0 | 100 | 1 μg/ml |
| 10 | 90 | 100 | 100 ng/ml |
| 1 | 99 | 100 | 10 ng/ml |
| 0.1 | 99.9 | 100 | 1 ng/ml |
| 0 | 100 | 100 | blank |

DNA Extraction from Maternal CD45+ Cells and Placental Tissue

DNA extraction solution (1 M Tris-HCl pH 8.0, 0.5M EDTA, sterile water, Proteinase-K, 10% SDS) was added to maternal CD45+ cells/minced placental tissue and incubated at 50° C. overnight. Supernatant was transferred to a fresh tube and equal volume of Phenol:Chloroform (Invitrogen) is added. The solution was vortexed until milky and centrifuged at 2095×g for 10 min. Supernatant was collected into a fresh tube and equal volume of isopropanol was added. The tube was inverted gently until DNA thread was visible. DNA thread was washed twice with 70% ethanol at 13,000 rpm for 2 min and left to dry. DNA was re-suspended in 100 μl of TE buffer.

### Real-Time PCR for Foetal Gender Determination

Real-time PCR analysis was done using PE Applied Biosystems 7000 Sequence Detector (Applied Biosystems, California, USA). Beta-globin (HBB), an endogenous control and a male sex determining region Y (SRY) foetal gender determination were analysed. The following SRY and HBB primers and probes (AIT Biotech, Singapore) were used: SRY-forward, 5′-TGG CGA TTA AGT CAA ATT CGC-3′ (SEQ ID NO:1); SRY-reverse, 5′-CCC CCT AGT ACC CTG ACA ATG TAT T-3 (SEQ ID NO:2)′; SRY-probe, 5′-(6-FAM) AGC AGT AGA GCA GTC AGG GAG GCA GA (TAMRA) (SEQ ID NO:3); HBB-forward, 5′-GTG CAC CTG ACT CCT GAG GAG A-3′ (SEQ ID NO:4); HBB-reverse, 5′-CCT TGA TAC CAA CCT GCC CAG-3′(SEQ ID NO:5); HBB-probe, 5′-(VIC) AAG GTG AAC GTG GAT GAA GTT GGT GG (TAMRA)-3′(SEQ ID NO:6). Commercial male genomic DNA (Promega) with known initial concentrations was serially diluted (5-folds) to generate the standard curves for HBB and SRY. Samples and standards were run in triplicates in the same assay. Water blanks were included in triplicates for each PCR as amplification negative controls. Reactions were set up in a reaction volume of 25 μl using the TaqMan Universal PCR Master Mix (Applied Biosystems). SRY and HBB primers were used in a final concentration of 450 nM and probes at a final concentration of 225 nM respectively. Three microliters of WGA product and 60 ng of genomic DNA were used for amplification. Thermal cycling for both SRY and HBB was initiated with a 2 min incubation at 50° C., to allow the uracil N-glycosylase (UNG) to act, followed by a first denaturation step of 10 min at 95° C. and then 55 cycles of 95° C. for 15 s and 60° C. for 1 min.

### Quantitative Fluorescence-PCR for Amelogenin (AMXY)

A QF-PCR analysis for AMXY short tandem repeat (STR) marker was performed in 25 μl reaction volume containing 5 ng of genomic DNA or 3 μl (WGA product), 0.1-0.4 μmoles of each fluorescent-labeled and unlabeled primer (AIT Biotech) and 1×PCR multiplex master mix (Qiagen, Hilden, Germany). One microlitre of the amplified allelic fragments was mixed with 9.5 μl formamide and 0.5 μl Genescan-500 Rox (6-carboxy-X-rhodamine) (Applied Biosystems) size standards in 96-well-reaction plate before denaturation at 95° C. for 2 min, and cooling at 4° C. for 2 min, for capillary electrophoresis with an ABI Prism 310 Genetic Analyser (Applied Biosystems) and analysis using GeneScan Analysis Software version 3.1 (Applied Biosystems).

Fluorescence In Situ Hybridization (FISH) of Micromanipulated FNRBCs from Maternal Blood

FNRBCs from CD147 sorted maternal blood as well as corresponding villi (control) were micromanipulated onto glass slides pre-marked with a diamond scribe to indicate location. FNRBCs (range: 8-15 FNRBCs) were loaded per slide. Slides were cytopsun at 700 rpm for 5 minutes and observed under light microscope to confirm location and number of FNRBCs before initiating FISH procedure. FNRBCs were fixed by adding Carnoys fixative (3:1 methanol:acetic acid) drop-by-drop on a thermal block at 60° C. Slides were immersed in 2× saline sodium citrate (SSC; 3M NaCl, 0.3M sodium citrate, pH7.0) and incubated at 37° C. for 30 min. Next, the slides are incubated at 37° C. for 13 min with pepsin (0.005%) diluted in 10 mM HCl. Slides are washed with 1×PBS for 5 min at room temperature. Slides were placed in fixation solution of 1% formalin and incubated for 5 min at room temperature and washed again with 1×PBS. The slides were dehydrated by placing them consecutively in increasing ethanol concentration (70%, 85%, 100%) for 1 min each at room temperature. Slides were incubated for 5 min in denaturing solutions containing 70% formamide (pH7).

Slides were then placed in ice-cold 70% ethanol and subsequently dehydrated with 90% and 100% ethanol. The Vysis CEP spectrum orange X and spectrum Y green probe (Vysis AneuVysion Multicolor DNA Probe Kit, Abbott Laboratories, Abbott Park, Ill., USA) was used in all samples. The CEP probe was mixed with hybridization buffer (50% formamide and 10% dextran sulphate in 2×SSC, pH 7.0) in the ratio of 2:3. Three microlitre of probe was added to each slide. The LSI probe was used directly. The slides were covered with a coverslip and the slides sealed with parafilm. Target DNA was denatured on an in situ hybridization block (MJ Research, Waltham, USA) at 75° C. for 7 min followed by hybridization at 37° C. for 16 h. The coverslip was removed prior to post-hybridization washes with 0.4×SSC/0.3% (NP)-40 at 73° C. for 2 min and 2×SSC/0.1% (NP)-40 at room temperature for 2 min. The slides were air dried at room temperature before mounting with 6 μl DAPI (4,6-diamidino-2-phenylindole) as counterstain. A 22 mm glass coverslip was mounted to spread the DAPI evenly before sealing with nail varnish. The fluorescence signals were visualized and enumerated under single and dual bandpass filter sets (DAPI, green, orange, aqua) (Abbott Laboratories) within an epifluorescence microscope (BX51, Olympus America Inc., Center Valley, Pa., USA) that was fitted with camera (Applied Imaging, Grand Rapids, Mich., USA) for image captured. Images were captured using FISHView, 2.0 EXPO (ASI, Carlsbad, USA).

### Statistics

Parametric statistics used: mean, standard deviation (SD), sample size (n) standard error (SE), standard error of mean (SEM), 95% confidence interval (CI), one way analysis of variance (ANOVA) and Bonferroni post-correction analysis. For non-parametric statistics used Wilcoxon signed rank, Mann-Whitney “U” and Linear Regression analysis. SPSS statistical software (SPSS Inc. USA) and GraphPad (GraphPad Software Inc., California, USA) used were used for statistical analysis of data.

### Results

### Determination of CD147 Antibody Dilution for Use in MACS Cell Sorting

CD147 antibody binds to both FNRBCs and adult RBCs. Binding of anti-CD147 antibody to adult RBCs is variable, though generally weaker than FNRBCs. Therefore, antibody titration experiments were carried out to determine the optimum concentration of antibody needed. Criteria for selection of an optimal antibody dilution for future experiments: 1) maximum recovery of FNRBCs in CD147 positive fraction (CD147+), 2) minimum recovery of FNRBCs in the negative fraction (CD147−), and 3) maximum depletion of adult RBCs from the positive fraction (CD147+).

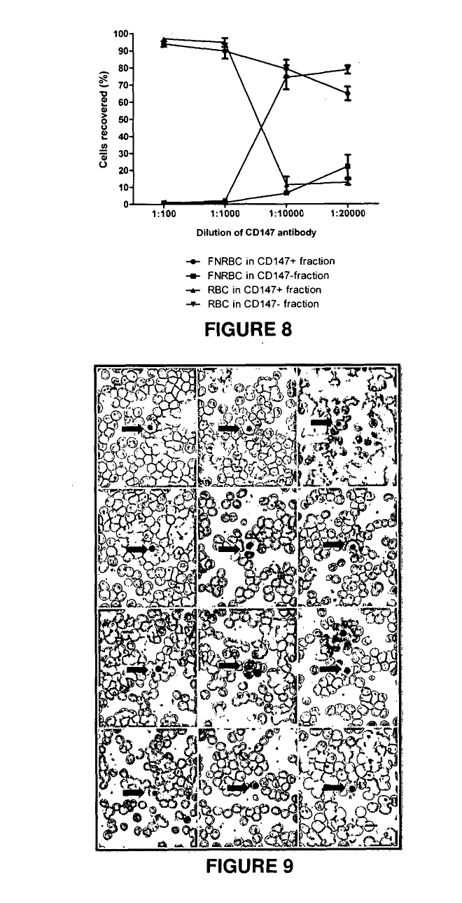

For antibody concentration of 1:100, mean recovery of FNRBCs from the CD147+ fraction and CD147− fraction was 94.0±1.6% (SD=2.8) and 1.0±0.1% (SD=0.3) respectively (FIG. 8). Mean recovery of adult RBCs in CD147+ fraction was 97±1.3% (SD=2.2) which is only a 3% depletion of adult RBCs (FIG. 8). For antibody concentration of 1:1,000, mean recovery of FNRBCs from the CD147+ fraction and CD147− fraction was 90±4.4% (SD=7.7) and 1.0±0.3% (SD=0.6) respectively. Depletion of adult RBCs from CD147+ fraction was 5% with a mean recovery of 95±2.5% (SD=4.4) (FIG. 8). Antibody concentration of 1:10,000 had mean recovery of FNRBCs from the CD147+ fraction and CD147− fraction 79.6±5.2% (SD=9.0) and 6.4±1.0% (SD=1.8) respectively. Mean recovery of adult RBCs from CD147+ fraction was 11.3±4.8 (SD=8.4) which resulted in 88.7% depletion of adult RBCs (FIG. 8). For antibody concentration of 1:20,000, mean recovery of FNRBCs from the CD147+ fraction and CD147− fraction was 65±4.0% (SD=7.0) and 22.3±6.8% (SD=11.8) respectively. Depletion of adult RBCs from CD147+ fraction was 87.1% (12.9±1.6% SD=2.7), similar to 1:10,000 (FIG. 8).

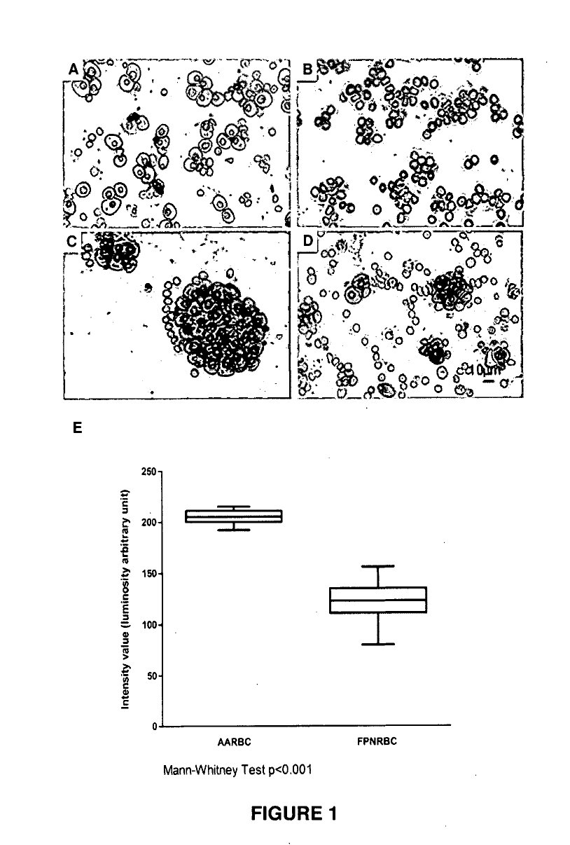

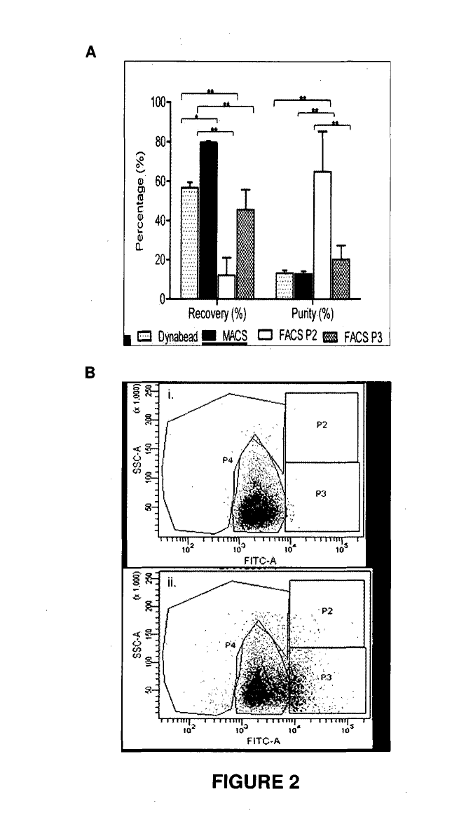

The results for the antibody concentration of 1:10,000 were satisfactory among the 4 antibody concentrations tested. Concentration of 1:10,000 met the 3 criteria: maximum recovery of FNRBCs in CD147+, minimum recovery of FNRBCs in CD147−, and maximum depletion of adult RBCs from CD147+. Antibody concentration 1:10,000 and 1:20,000 had similar depletion rates for adult RBCs, however, recovery of FNRBCs from CD147+ fraction was lower in 1:20,000. FNRBCs expressed higher amounts of CD147 antigen on its surface (FIG. 1) therefore, even with diluted antibody concentration (1:10,000), allowed anti-CD147 antibody to bind to CD147 surface antigens present on FNRBCs much more easily and readily. In adult RBCs the expression of surface antigen CD147 was much lower than that of FNRBCs (FIG. 1), thus, less likely to bind to anti-CD147 antibody.

Enrichment of e+FNRBCs from Maternal Blood Collected Prior to Termination of Pregnancy (TOP) Procedure

To determine if a protocol using anti-CD147 antibody can enrich foetal primitive erythroblasts (FNRBCs) from maternal blood samples obtained prior to first trimester termination of pregnancy, peripheral blood (15-20 ml; n=20) was taken immediately prior to elective termination of pregnancy procedure, and processed without delay. The blood was diluted 1:3 with PBS, layered over Percoll™ 1118 and centrifuged. The interface layers were collected and negative depletion of CD45+ cells/CD147 positive selection of cells performed. Cells were cytospun onto glass slides. The slides were Wright's stained for morphological identification of FNRBCs. Slides with identified FNRBCs were de-stained and immunocytochemistry for the identification of epsilon-globin in these cells. Epsilon positive FNRBCs (1,3,5 e+FNRBCs) were laser captured, microdissected and catapulted into PCR tubes. Cells were amplified by whole genome amplification and array comparative genomic hybridization performed.

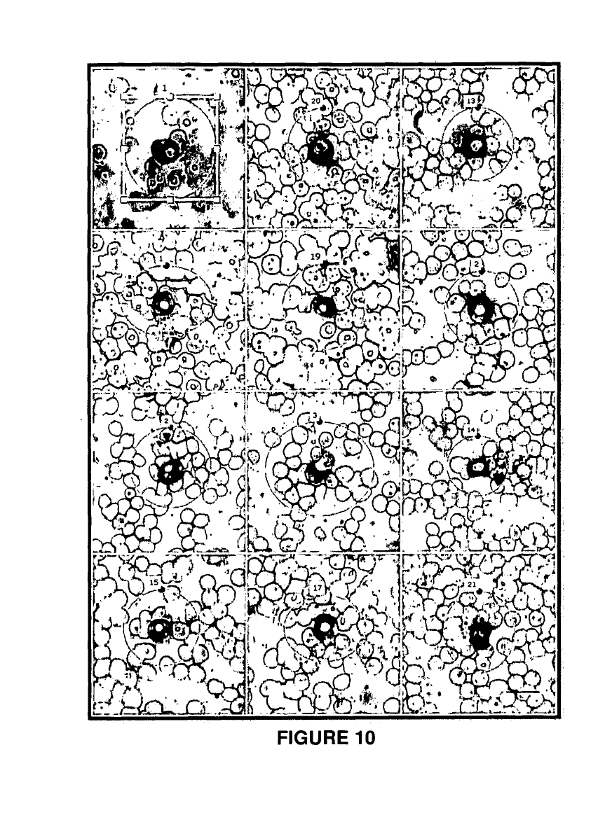

Cells cytospun onto slides were Wright's stain and FNRBCs identified morphologically (FIG. 9). The slides that contained FNRBCs were de-stained and immunocytochemistry for epsilon-globin showed that FNRBCs were foetal in origin (FIG. 10). The mean recovery of e+FNRBCs was 25.1±22.9 per sample (Table 5). e+FNRBCs recovered in all samples which ranged between 1-105 cells; approximately 1.32 e+FNRBCs per ml of maternal blood obtained at a mean gestational age of 9±0.8 weeks (Table 5). One maternal blood sample had higher numbers of e+FNRBCs (55 e+FNRBCs vs 19 e+FNRBCs in euploid cases; p>0.05) came from a pregnant woman carrying an abnormal foetus with 46,XXX (as confirmed by karyotype later). Another sample which had the highest numbers of e+FNRBCs (105 cells) had a normal karyotype however, upon closer observation of maternal clinical history did not reveal any clinical cause except for full blood count demonstrating microcytic, hypochromic anaemia. This may have caused the foetus to mount a physiological response to chronic hypoxia by rapid production of nucleated red blood cells.

TABLE 5
Enrichment of e+FNRBCs after density gradient
centrifugation and CD45/CD147 sorting from
pre-TOP maternal blood (n = 20).

| Maternal age (mean ± SD) | 27.3 ± 8.3 years |
|---|---|
| Gestational age of foetus (mean ± SD) | 9.0 ± 0.8 weeks |
| Total volume of blood processed (ml) | 380 (range: 10-20) |
| Total e⁺FNRBC enriched | 502 (range: 1-105* cells/sample) |
| e⁺FNRBC/sample | 25.1 ± 22.9 (19.0 ± 10.3)** |
| e⁺FNRBC/ml | 1.32 (1.0)** |

*patient with clinical history of anaemia and abnormal foetus 69,XXX.
**exclusion of e+FNRBCs from patient with clinical history of anaemia and abnormal foetuses 46,XXX

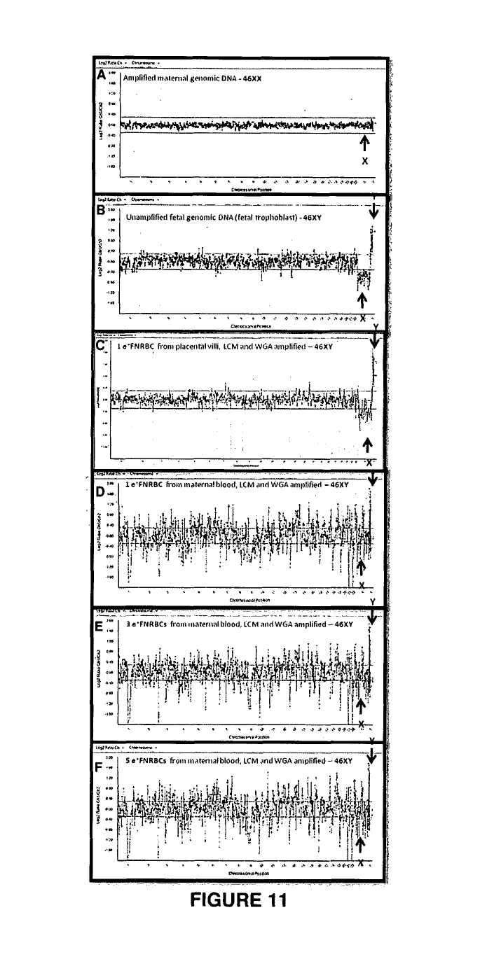

These e+FNRBCs can be laser capture microdissected and array CGH for genetic diagnosis performed. Preliminary data of CD147+ enriched epsilon-globin positive FNRBCs from a pre-TOP maternal blood sample in a pregnancy with a male foetus (FIG. 11). The e+FNRBCs were laser microdissected (LCM) and catapulted into a PCR tube. DNA material from these cells (1,3,5 FNRBCs) were amplified separately by whole genome amplification (WGA) and microarray-based comparative genomic hybridization (aCGH) were performed. Arrays profiles of 1, 3 and 5 FNRBCs confirm that the cells are foetal in origin by the presence of the XY signal (FIG. 11D,E,F).

Consistent enrichment and the identification of e+FNRBCs was achieved in all samples. This data shows that FNRBCs were present in first trimester maternal blood and their identity was confirmed by immunophenotyping with an intracellular monoclonal antibody against haemoglobin-epsilon. Others have enriched FNRBCs from maternal blood, however the methods used to confirm FNRBCs identity were mainly based on morphology which do not confirm foetal identity. The recovery of 1-1.3 e+FNRBCs per ml of maternal blood was in concordance with earlier reports using other strategies to confirm foetal identity. Enrichment of e+FNRBCs from maternal blood samples was also attempted by others with inconsistent recoveries.

Of the 20 samples, 1 abnormal pregnancy was detected (confirmed later by karyotype) of a foetus with 69,XXX. A total of 55 e+FNRBCs were enriched from maternal blood of the triploidy pregnancy much higher than the mean of 19 e+FNRBCs per sample in normal pregnancies. Foetal cells have been reported to be more in maternal blood circulation in aneuploid pregnancies. This may be secondary to the increased trafficking of foetal cells into the maternal blood in the presence of chromosomal abnormalities in the foetus or abnormalities of the placental barrier.

These e+FNRBCs can be laser capture microdissected and array CGH for genetic diagnosis performed. Preliminary results are promising; however, further optimization is required to reduce the noise-to-signal ratio in order to make genetic calls easy and accurate before being offered as a diagnostic service.

Enrichment of FNRBCs from First Trimester Pre-Termination of Pregnancy Maternal Blood, Manual Collection Using Micromanipulator and Downstream Molecular Analyses

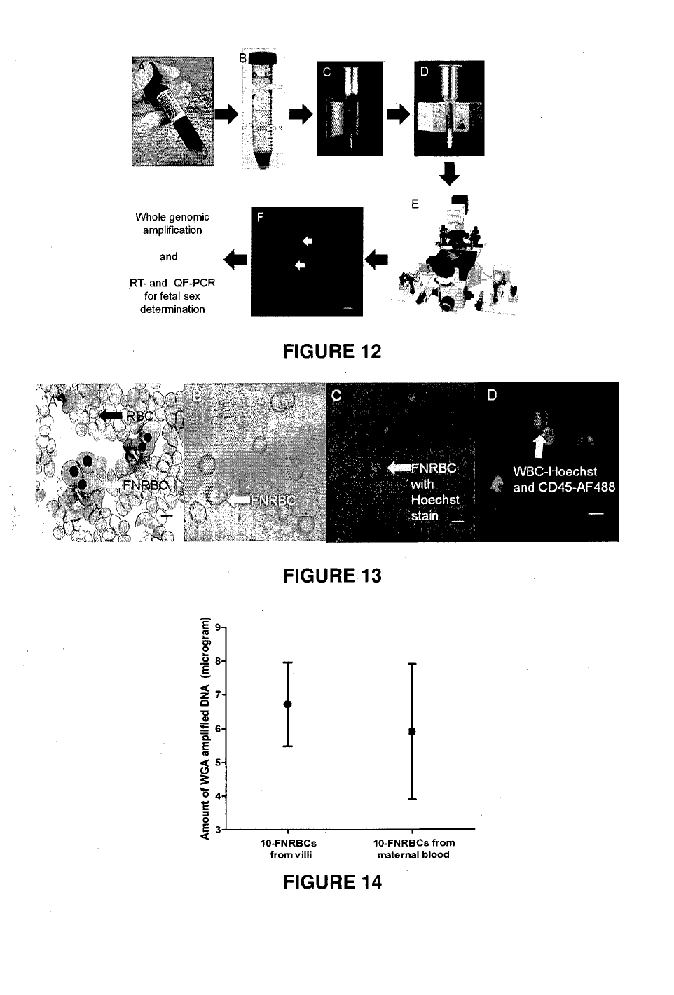

To determine if a protocol using anti-CD147 antibody can enrich foetal primitive erythroblasts (FNRBCs) from maternal blood samples obtained prior to first trimester termination of pregnancy and are foetal in origin, peripheral blood (10-20 ml; n=8) samples were collected immediately prior to elective termination of pregnancy procedure, and processed immediately. The blood was diluted 1:3 with PBS, layered over Percoll™ 1118 and centrifuged. The interface layers were collected and negative depletion of CD45+ cells and CD147 positive selection of cells performed. Cells from CD147+ fraction were stained with Hoechst and CD45-AF488 and manually picked using a micromanipulator (FIG. 12). FNRBCs were picked in groups of 10 cells whenever possible.

The results showed that FNRBCs could be differentiated from other nucleated cells in the CD147+ fraction based on the presence of nuclear staining (Hoechst+), absence of surface antigen CD45 (CD45-AF488-) and morphology. FNRBCs enriched from pre-TOP maternal blood samples (n=8) were identified by fluorescent staining. Morphologically, FNRBCs have higher cytoplasmic:nuclear ratio (FIG. 13A), and upon fluorescent staining in solution, FNRBCs could be identified and manually picked using a micromanipulator for transfer to PCR tubes (FIG. 12E): FNRBCs were CD45-negative and Hoechst stained (blue) under fluorescent microscope (shown is grey in FIG. 13C). Mononuclear WBCs stained positive for both anti-CD45-AF488 and Hoechst (FIG. 13D).

This strategy recovered a mean 53±17.3 FNRBCs (range: 8-151 cells) (Table 6). Cells were recovered in each consecutive sample and approximately 2.7 FNRBCs per ml of maternal blood was obtained at a mean gestational age of 8.7±0.9 weeks (Table 6). All foetuses had normal karyotype except for one with Trisomy 3 (39 FNRBCs). Two samples had higher than expected numbers of FNRBCs, 103 and 151 respectively, however, no obvious clinical cause could be found to explain the high numbers of FNRBCs obtained from maternal blood in these two cases.

TABLE 6
Enrichment of FNRBCs after density gradient centrifugation and
CD45/CD147 sorting from pre-TOP maternal blood (n = 8)

| Maternal age (mean ± SD) | 30.9 ± 8.4 years |
|---|---|
| Gestational age of foetus (mean ± SD) | 8.7 ± 0.9 weeks |
| Total volume of blood processed | 156 (range: 17-20 ml) |
| Total FNRBCs enriched | 424 (range: 8-151 cells/sample) |
| FNRBCs/sample (mean ± SD) | 53 ± 17.3 |
| FNRBCs/ml | 2.7 |

Consistent enrichment and micromanipulation of FNRBC (Hoechst+/CD45-AF488-) was observed in all 8 maternal blood samples. An average of 2.7 FNRBCs per ml of maternal blood (range 8-151 cells/sample) was recovered by fluorescent staining and micromanipulation. Others have reported success in retrieving FNRBCs with micromanipulation and most have recovery rates which are similar to data presented above. Another report using micromanipulated cells identified using epsilon haemoglobin confirmed the gender by QF-PCR in 91% of the cases. Micromanipulation was also used to collect foetal cells to diagnose abnormal pregnancies including Duchenne's muscular dystrophy, rhesus D and haemoglobinopathies. An average of 3.2 foetal cells from 7 ml maternal blood (1-7 cells per sample) was obtained to diagnose Duchenne muscular dystrophy. Rhesus D was diagnosed using cells isolated from 4 cases of post-termination maternal blood (1.8 FNRBCs/ml of maternal blood). Others retrieved an average of 1.4 FNRBCs/ml of maternal blood (7-22 cells per sample) to diagnose haemoglobinopathies such as sickle cell anaemia and β-thalassaemia.

In the present study, FNRBCs (Hoechst+/CD45-AF488-) collected by micromanipulation in 8 maternal blood samples were used for further confirmation of their foetal origin.

Confirmation of Foetal Origin of Micromanipulated FNRBCs Enriched from Maternal Blood

### A. Whole Genome Amplification

Micromanipulator assisted picking of FNRBCs enriched from maternal blood after anti-CD147 antibody separation was based on morphology and nuclear stain (Hoechst). For confirmation that manipulated cells are indeed foetal in origin, prior amplification of the FNRBC genetic material was needed for downstream molecular analyses. To perform whole genome amplification of FNRBCs and assess the yield of amplified material.

The FNRBCs obtained using the above protocol was collected in groups of 10 FNRBCs per tube. Whole genome amplification of the FNRBC samples (n=8) and picogreen measurements of DNA yield post-amplification were performed. The yield was compared to DNA amplified from 10 FNRBCs manually picked from villi tissue (control).

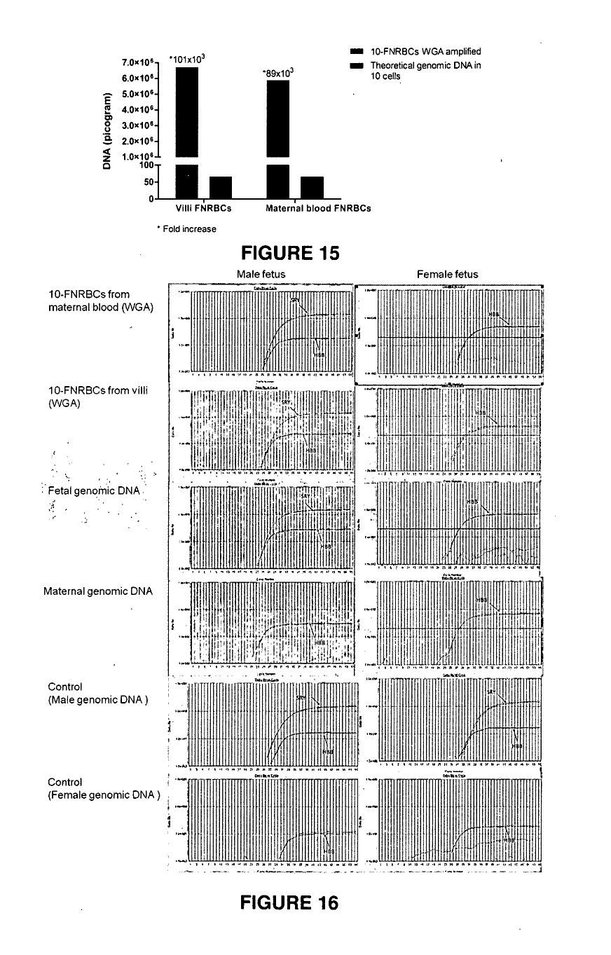

The results showed that the mean DNA yield for 10-FRNBCs from maternal blood was 5.9±0.8 μg (mean±SEM) (SD=2.4; 95% CI 3.9-7.9) (FIG. 14) which is an 89×103-fold more than that present in theoretical calculated genomic DNA in 10 cells (FIG. 15). The mean DNA yield for 10-FNRBCs from villi was 6.7±0.5 μg (mean±SEM) (SD=1.5; 95% CI 5.5-7.9) which is a 101×103-fold increase compared that present in theoretically calculated genomic DNA in 10 cells (FIG. 15). Although the mean DNA yield from villi obtained 10-FNRBCs was higher than that from maternal blood no observed significant difference was seen (p>0.05) (FIG. 14).

The DNA yield after whole genome amplification of FNRBCs manually picked from maternal blood was comparable to the DNA yield of the FNRBCs from picked from villi which served as a control. Whole genome amplification increased the amount of genetic material approximately 90-fold therefore enabling downstream molecular analyses.

### B. Real-Time Polymerase Chain Reaction (PCR) for Foetal Sex Determination

To determine the foetal origin of micromanipulated FNRBCs enriched from maternal blood by real-time PCR using a male sex determining region Y (SRY), real-time PCR for SRY and human beta-globin (HBB, an endogenous control) was performed using 3 μl the WGA amplified DNA from FNRBCs (n=8). As controls, amplified DNA from FNRBCs manually picked from villi, foetal genomic DNA (extracted from trophoblast tissue), maternal genomic DNA (extracted from CD45+ WBCs), male genomic DNA and female genomic DNA were also analysed in each experiment.

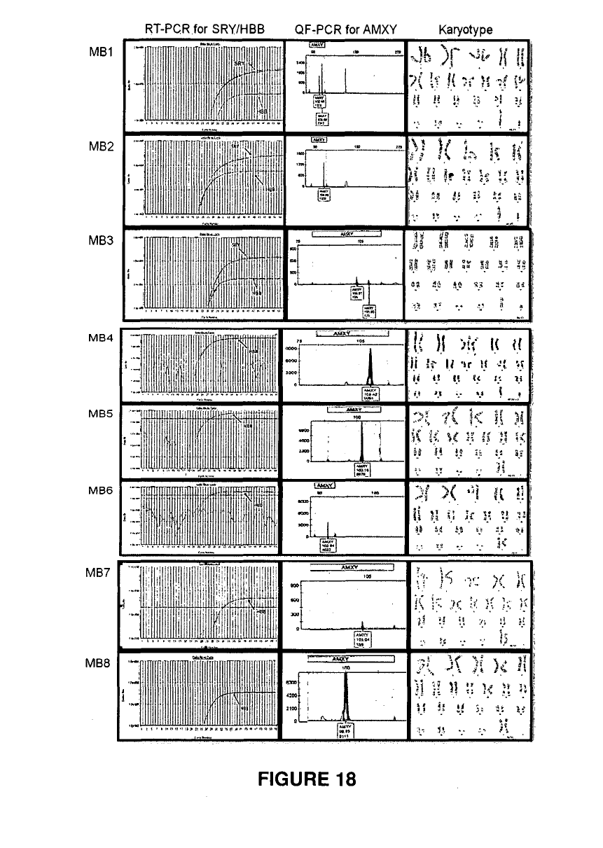

The results showed that genetic confirmation of FNRBCs obtained was foetal in origin. FNRBCs enriched from pregnancies carrying male foetus were confirmed by real-time PCR for SRY (75%) whereas FNRBCs from pregnancies carrying female foetus were determined by absence of SRY and presence of HBB (100%) (FIG. 16, Table 7). Foetal gender in seven of the eight cases were predicted accurately (FIG. 18; Table 8).

TABLE 7
Presence of SRY and HBB in FNRBCs enriched from maternal
blood in pregnancies carrying male and female foetuses.

| | SRY | HBB |
|---|---|---|
| Male (n = 4) | 75% | 100% |
| Female (n = 4) | 0 | 100% |

The FNRBCs enriched using anti-CD147 antibody and micromanipulated were confirmed as foetal in origin except in one case. Amplification of SRY by real-time PCR of FNRBCs from one of the four samples with male pregnancy was not successful even though the AMELY gene (amelogenin Y) was shown to be present for the same sample by quantitative fluorescence PCR (QF-PCR) indicating that as a male. This is possibly due to the problem of allelic dropout (ADO) unique to PCR of small quantities of DNA. The precise cause of ADO remains unknown. ADO most likely arises in the initial cycles of primary PCR before the target molecules undergo amplification. Suggestions that ADO maybe from 1) suboptimal PCR conditions, 2) incomplete cell lysis, 3) inadequate separation of both DNA strands, 4) DNA strand breaks, 5) DNA damage or deterioration during preparation, 6) restricted access to the target genomic sequence (G/C rich regions) by the primers and Taq polymerase reduces denaturation efficiency observed <40% PCR success rate of FNRBCs isolated from maternal blood used to diagnose β-thalassaemia as possibly arising from the apoptotic nature of these cells and condensed nucleus. Single cell amplification has high rates of ADO and some researchers have suggested a minimum of three cells is needed to achieve 100% amplification efficiency and ADO rates that approach zero.

### C. Quantitative Fluorescent-PCR for Amelogenin (AMXY)

To determine that micromanipulated FNRBCs recovered from maternal blood are of foetal in origin by quantitative fluorescent PCR using amelogenin (AMXY) foetal gender determination. Amelogenin gene is a single copy gene located on Xp22.1-Xp22.3 (AMELX) and Yp 11.2 (AMELY). AMELX gives a 103 bp ampicon whereas AMELY gives rise to 108 bp amplicon. QF-PCR for amelogenin (AMXY) was performed on the amplified DNA from FNRBCs (n=8). As controls, amplified DNA from FNRBCs manually picked from villi and maternal genomic DNA were analysed.

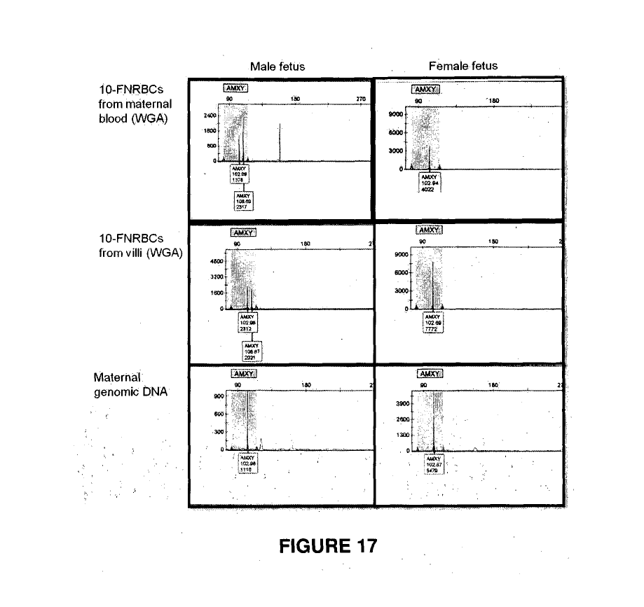

The results show that genetic confirmation of FNRBCs obtained were of foetal in origin by QF-PCR analysis. Male foetal cells obtained from maternal blood carrying male foetus were confirmed by two alleles (103 and 108) on amelogenin (AMXY), whereas female foetal cells obtained from maternal blood carrying female foetus were determined by a single allele (103) for AMXY (FIG. 17). In MB2 and MB4 (FNRBCs from maternal blood carrying male foetus), only the 108 allele (AMELY gene) was amplified and allelic dropout was observed for the AMELX. However, genders for all 8 cases were predicted accurately (FIG. 18).

TABLE 8
QF-PCR for amelogenin of FNRBCs from maternal blood.

| | AMELX | AMELY |
|---|---|---|
| | (103 bp amplicon) | (108 bp amplicon) |
| Male foetus (n = 4) | 50% | 100% |
| Female foetus (n = 4) | 100% | 0% |

QF-PCR analysis of FNRBCs enriched using anti-CD147 antibody and micromanipulated was able to prove that cells belong to the foetuses and respective sexes as confirmed by karyotyping. In two of the eight cases ADO was seen with the failure of AMELX amplification in male foetuses however, AMELY gene was successfully amplified in both cases, which confirmed their sex as males.

D. Fluorescence In Situ Hybridization (FISH) of Micromanipulated FNRBCs from Maternal Blood

To perform fluorescence in situ hybridization (FISH) of micromanipulated FNRBCs from maternal blood FNRBCs micromanipulated from 6 post-termination of pregnancy (TOP) maternal blood and 1 pre-TOP maternal blood was tested. Corresponding villi FNRBCs were micromanipulated onto glass slides as controls.

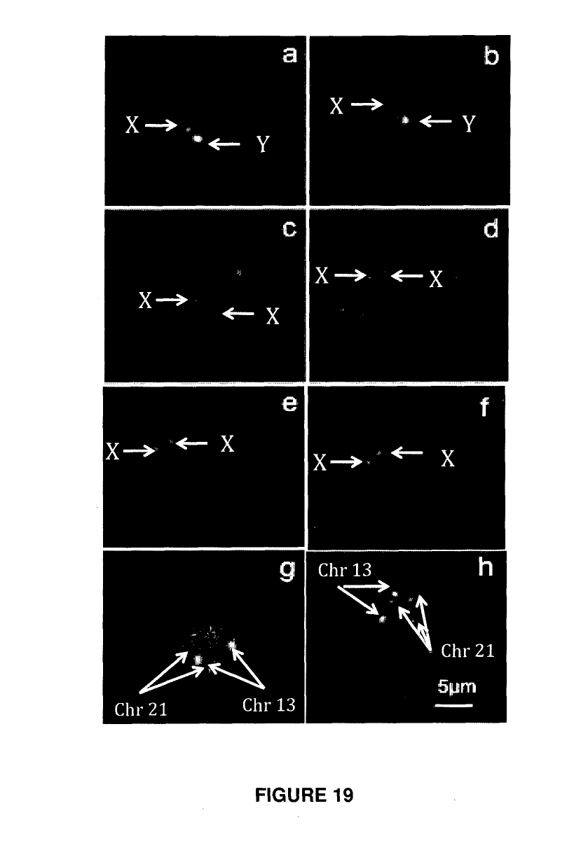

The results show that CEP XY signals were seen in at least one FNRBC in all post- and pre-TOP samples. The number of positive signals per sample varied (Table 9). The FISH signals are shown in FIG. 19.

TABLE 9
Summary of FISH on micromanipulated FNRBCs from
post- and pre-termination of pregnancy maternal blood

| | | | | | FNRBC - | |
|---|---|---|---|---|---|---|
| | | | | | no. of FISH | |
| | | Gestation | | Gender | signals | |
| Samples | Type | (Weeks) | Probe | (FISH villi) | MB | Villi |
| MB74 | Post-TOP | 8 + 3 | CEP | Male | 4/10 | 8/10 |
| | | | X/Y | | | |
| | Post-TOP | 8 + 3 | LS1 | Male | 1/3¶ | — |
| | | | 13/21 | | | |
| MB76 | Post-TOP | 9 + 1 | CEP | Male | 15/15 | 5/7 |
| | | | X/Y | | | |
| MB79 | Post-TOP | 9 + 3 | CEP | Male | 2/2° | 6/10 |
| | | | X/Y | | | |
| MB84 | Post-TOP | 8 + 1 | CEP | Female | 8/8 | 7/7 |
| | | | X/Y | | | |
| | Pre-TOP | 8 + 1 | CEP | Female | 1/2* | 7/7 |
| | | | X/Y | | | |
| MB85 | Post-TOP | 8 + 5 | CEP | Female | 9/9 | 9/9 |
| | | | X/Y | | | |

¶MB74 - 10 cells were micromanipulated on the slide, 3/10 cells were located after FISH and 1 of 3 showed chromosome 13, 21 signal.
°MB79 - 10 cells were loaded onto slide but only 2 cells were located post-FISH and both had positive FISH signals.
*MB84 - 10 cells were loaded onto glass slide but only 2 cells were located post-FISH. One of two showed clear FISH signals.

### Conclusion

The anti-CD147 antibody has been shown to be useful for consistent enrichment of e+FNRBCs from maternal blood, and FNRBCs could reliably be identified and collected for amplification by WGA and suitable for genetic interrogation by real time PCR and quantitative fluorescent-PCR. Micromanipulated FNRBCs from maternal blood have also been shown to be amenable to fluorescence in situ hybridization. Together these findings have demonstrated the potential use of anti-CD147 antibody for enrichment of FNRBCs from maternal blood for first trimester non-invasive prenatal diagnosis.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

1. Ponnusamy S, Mohammed N, Ho S S, et al. In vivo model to determine fetal-cell enrichment efficiency of novel noninvasive prenatal diagnosis methods. Prenat Diagn. 2008; 28:494-502.
2. Sambrook and Green, Molecular Cloning: A Laboratory Manual, Cold Springs Harbor Laboratory (Fourth Edition), New York (2012).
3. Steele M W, Breg W R Jr. Chromosome analysis of human amniotic-fluid cells. Lancet 1966; 1(7434):383-5.
4. Choolani M, O'Donoghue K, Talbert D, et al. Characterization of first trimester fetal erythroblasts for non-invasive prenatal diagnosis. Mol Hum Reprod. 2003; 9:227-235.
5. Bhat N M, Bieber M M, Teng N N. One-step enrichment of nucleated red blood cells. A potential application in perinatal diagnosis. J Immunol Methods. 1993; 158:277-280.
6. Bianchi D W, Klinger K W, Vadnais T J, et al. Development of a model system to compare cell separation methods for the isolation of fetal cells from maternal blood. Prenat Diagn. 1996; 16:289-298.
7. Bianchi D W, Zickwolf G K, Weil G J, Sylvester S, DeMaria M A. Male fetal progenitor cells persist in maternal blood for as long as 27 years postpartum. Proc Natl Acad Sci USA. 1996; 93:705-708.
8. Ganshirt D, Smeets F W, Dohr A, et al. Enrichment of fetal nucleated red blood cells from the maternal circulation for prenatal diagnosis: experiences with triple density gradient and MACS based on more than 600 cases. Fetal Diagn Ther. 1998; 13:276-286.
9. Jansen M W, Korver-Hakkennes K, van Leenen D, et al. How useful is the in vitro expansion of fetal CD34+ progenitor cells from maternal blood samples for diagnostic purposes? Prenat Diagn. 2000; 20:725-731.
10. Troeger C, Holzgreve W, Hahn S. A comparison of different density gradients and antibodies for enrichment of fetal erythroblasts by MACS. Prenat Diagn. 1999; 19:521-526.
11. Smits G, Holzgreve W, Hahn S. An examination of different Percoll density gradients and magnetic activated cell sorting (MACS) for the enrichment of fetal erythroblasts from maternal blood. Arch Gynecol Obstet. 2000; 263:160-163.
12. Voullaire L, loannou P, Nouri S, Williamson R. Fetal nucleated red blood cells from CVS washings: an aid to development of first trimester non-invasive prenatal diagnosis. Prenat Diagn. 2001; 21:827-834.
13. Seppo A, Frisova V, lchetovkin I, et al. Detection of circulating fetal cells utilizing automated microscopy: potential for noninvasive prenatal diagnosis of chromosomal aneuploidies. Prenat Diagn. 2008; 28:815-821.
14. Wachi T, Kitagawa M. Studies on preliminary concentration methods for recovery of fetal nucleated red blood cells in maternal blood. Congenit Anom (Kyoto). 2004; 44:196-203.
15. Shinya M, Okamoto A, Sago H, et al. Analysis of fetal DNA from maternal peripheral blood by lectin-polymerase chain reaction-single strand conformation polymorphism. Congenit Anom (Kyoto). 2004; 44:142-146.
16. Krabchi K, Gadji M, Samassekou O, Gregoire M C, Forest J C, Drouin R. Quantification of fetal nucleated cells in maternal blood of pregnant women with a male trisomy 21 fetus using molecular cytogenetic techniques. Prenat Diagn. 2006; 26:28-34.
17. Kilpatrick M W, Tafas T, Evans M I et al. Automated detection of rare cells in maternal blood: eliminating the false-positive XY signals in XX pregnancies. Am J Obstet. Gynecol. 2004; 190(6):1571-8.
18. Percoll™ Methodology and Applications 2nd Edition, Amersham Pharmacia Biotech, UK

1. A method of detecting, separating and/or isolating at least one foetal nucleated red blood cell (FNRBC) from a sample comprising:
a) treating the sample using centrifugation in a density gradient medium comprising of a density selected from 1.077-1.120 g/ml;
b) contacting the sample with an antibody or antigen binding fragment thereof that specifically binds CD147 at a concentration of at least 1:20,000, thereby producing a mixture;
c) maintaining the mixture under conditions in which a complex forms between the agent and the CD147 in the sample; and
d) separating the complex from the mixture;
thereby detecting, separating and/or isolating the FNRBC from the sample.

2. The method according to claim 1, wherein the concentration of the antibody or antigen binding fragment thereof is selected from at least 1:10,000, at least 1:1000 and 1:100.

3-4. (canceled)

5. The method according to claim 1, wherein the antibody is a polyclonal antibody, a monoclonal antibody, a chimeric antibody, a humanized antibody or a combination thereof.

6. The method according to claim 1, wherein the immune complex is separated from the mixture using immunomagnetic separation, flow cytometry or a combination thereof.

7. The method according to claim 1, wherein the sample is maternal blood, maternal tissue, cord blood or a combination thereof.

8. The method according to claim 7, wherein the maternal tissue is trophoblast tissue, liver tissue, placental tissue or a combination thereof.

9. The method according to claim 7, wherein about 1.16e+FNRBCs per milliliter of maternal blood is separated.

10. The method according to claim 7, further comprising contacting the maternal blood with an antibody or antigen binding fragment thereof that specifically binds to at least one marker on at least one white blood cell.

11. The method according to claim 10, wherein the marker is CD45, CD14 or a combination thereof.

12-15. (canceled)

16. The method according to claim 1, further comprising testing the FNRBC for sex discernment.

17. The method according to claim 16, wherein testing for sex discernment comprises determining presence or absence of an SRY gene, wherein if the SRY gene is present then the sample is from a foetus that is male and if the SRY gene is absent then the sample is from a foetus that is female.

18. The method of claim 17 wherein the SRY gene is detected using whole genome amplification and real time polymerase chain reaction (PCR).

19. The method according to claim 1, wherein the FNRBC is further tested for at least one prenatal disorder.

20. The method according to claim 19, wherein the prenatal disorder is selected from a chromosomal disorder, a genetic disorder, and a combination thereof.

21. The method according to claim 20, wherein the prenatal disorder comprises one or more of Down Syndrome, Edwards Syndrome, Patau Syndrome, a neural tube defect, spina bifida, cleft palate, Tay Sachs Disease, sickle-cell anemia, thalassemia, cystic fibrosis, fragile X syndrome, spinal muscular atrophy, myotonic dystrophy, Huntington's Disease, Charcot-Marie-Tooth disease, haemophilia, Duchenne muscular dystrophy, mitochondrial disorder, Hereditary multiple exostoses, and osteogenesis imperfecta.

22. The method according to claim 1, further comprising testing the FNRBC by one or more of fluorescent in situ hybridization (FISH), polymerase chain reaction (PCR), multiple ligand-dependent probe amplification (mpla), short tandem repeat analysis, array comparative genomic hybridization (CGH), genotyping, single plex sequencing, and massively parallel sequencing.

23. The method according to claim 1, wherein the FNRBC is from a mother in a first trimester, a second trimester or a third trimester of a pregnancy.

24. The method according to claim 1, wherein the FNRBC is a human FNBRC.

25. The method according to claim 1, wherein the sample comprises at least one anucleated red blood cell (ARBC).

26. The method according to claim 25, wherein the ARBC is an adult anucleated RBC (AARBC).


SEQUENCE LISTING (6 sequences)

SEQ ID NO: 1 - DNA, 21 bp, Artificial sequence
  SRY-forward primer
tggcgattaagtcaaattcgc

SEQ ID NO: 2 - DNA, 25 bp, Artificial Sequence
  SRY-reverse primer
ccccctagtaccctgacaatgtatt

SEQ ID NO: 3 - DNA, 26 bp, Artificial Sequence
  SRY-probe
agcagtagagcagtcagggaggcaga

SEQ ID NO: 4 - DNA, 22 bp, Artificial Sequence
  HBB-forward primer
gtgcacctgactcctgaggaga

SEQ ID NO: 5 - DNA, 21 bp, Artificial sequence
  HBB-reverse primer
ccttgataccaacctgcccag

SEQ ID NO: 6 - DNA, 26 bp, Artificial sequence
  HBB-probe
aaggtgaacgtggatgaagttggtgg